(19) United States
(12) Patent Application Publication
(10) Pub. No.: US 2023/0124115 A1
(43) Pub. Date: Apr. 20, 2023

(54) DELTA LACTONES THROUGH ENGINEERED POLYKETIDE SYNTHASES

(71) Applicant: THE REGENTS OF THE UNIVERSITY OF CALIFORNIA, Oakland, CA (US)

(72) Inventors: Jay D. KEASLING, Emeryville, CA (US); Amin ZARGAR, Emeryville, CA (US)

(21) Appl. No.: 18/053,288

(22) Filed: Nov. 7, 2022

Related U.S. Application Data
(63) Continuation of application No. PCT/US2021/031213, filed on May 7, 2021.
(60) Provisional application No. 63/021,554, filed on May 7, 2020.

(51) Int. Cl. C12P 7/62; C12N 9/00

(52) U.S. Cl. CPC C12P 7/62; C12N 9/93; C12Y 604/00

(57) ABSTRACT

Polyketide synthases are engineered to produce lactones. In the first module, an acyltransferase is swapped and in the second module a reductive loop is swapped. With another acyltransferase swap in the second module, we can programmably produce the non-methylated delta lactone.

### CROSS-REFERENCE TO RELATED APPLICATIONS

This application claims priority as a continuation application of PCT International Patent Application No. PCT/US2021/31213, filed May 7, 2021, which claims priority to U.S. Provisional Patent Application Ser. No. 63/021,554, filed May 7, 2020, both of which are hereby incorporated by reference.

### STATEMENT OF GOVERNMENTAL SUPPORT

The invention described and claimed herein was made utilizing funds supplied by the U.S. Department of Energy under Contract No. DE-AC02-05CH11231. The government has certain rights in this invention.

### FIELD OF THE INVENTION

The present invention is in the field of polyketides.

### BACKGROUND OF THE INVENTION

Polyketide synthase (PKS) engineering is an attractive method to generate new molecules such as commodity, fine and specialty chemicals. A significant challenge in PKS design is engineering a partially reductive module to produce a saturated β-carbon through a reductive loop exchange.

### SUMMARY OF THE INVENTION

In an aspect the invention provides methods of making delta-lactones comprising engineering lipomycin PKS to produce the delta-lactones. Engineered PKSs are used to construct dimethylated, single-methylated delta-lactones, and with facile manipulations, nonmethylated delta-lactones, which are valuable fragrances. In particular, we engineered the lipomycin PKS to produce triketide lactones. In the first module, we performed an acyltransferase swap with a BorAT and in the second module we performed a reductive loop swap with the NanA2 module. We performed two reductive loop swaps, from SpnB and NanA2, but only NanA2 worked. With another AT swap in the second module, we can programmably produce the non-methylated delta lactone. The methods employ a malonyl-CoA selecting analog in the first and second module, a KR only in the first module, and a full reductive loop in the second module. As the lipomycin PKS takes a variety of starters, the methods may be used to make delta lactones of varying size. Generation of lactones is of great industrial utility, and PKS engineering has been proposed (e.g. Kaus et al, Nat. Prod. Rep., 2018, 35, 1070) but successfully engineering lipomycin PKS to generate lactones was challenging, unexpected and unprecedented.

In embodiments:
the lactones are dimethylated delta-lactones, single-methylated delta-lactones, or nonmethylated delta-lactones;
the methods comprise, in a first module, performing an acyltransferase (AT) swap with a BorAT and in a second module performing a reductive loop swap with a NanA2 to programmably produce a single-methylated delta lactone;
the methods comprise, in a first module, performing an acyltransferase (AT) swap with a BorAT and in a second module performing a reductive loop swap with a NanA2, and another AT swap in the second module to programmably produce a non-methylated delta lactone; and/or
a malonyl-CoA selecting analog is employed in the first and second module, a KR only in the first module, and a full reductive loop in the second module.

In an aspect the invention provides related compositions including a composition comprising an engineered lipomycin PKS1 gene (or gene product) altered with an AT-swap from borreledin and a LipPKS2 altered with a donor reductive loop from NanA2, configured to produce a single-methylated lactone.

In embodiments the compositions comprise another AT swap on LipPKS2 from borreledin, configured to produce a non-methylated delta lactone.

The invention encompasses all combinations of the particular embodiments recited herein, as if each combination had been laboriously recited, such as recited herein.

Other objects, features, and advantages of the present invention will be apparent to one of skill in the art from the following detailed description and figures.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Experimental design of RL swaps. Conserved residues are identified through multiple sequence alignment surrounding the reductive domains (A, B and C). Donor RLs are inserted into the native lipomycin module 1, and the attached DEBS thioesterase hydrolyzes the product:

AT_{L} Loading acyltransferase
ACP_{L} Loading acyl carrier protein
AT Acyltransferase
KR Keto reductase
ER Enoylreductase
KS Ketosynthase
DH Dehydratase
TE Thioesterase (DEBS)
A Conserved residue upstream of loop
B Conserved residue upstream of loop
C Conserved residue downstream of loop

FIG. 2A. Phylogenetic and chemical similarity effects on reductive loop exchanges: Phylogenetic distance of the native LipPKS1 KR domain to each donor KR. The value above each bar denotes KR sequence identity comparison. FIG. 2B. AP (bar) and MCS (dots) chemical similarity between the native LipPKS1 KR domain and each donor KR. Chemical structures display native KR substrate in each module. FIG. 2C. Polyketide production of engineered PKSs at both junction “A” and junction “B” in biological triplicate.

FIG. 3A. A chemoinformatic approach to reductive loop exchanges: ClusterCad search revealed the closest substrates to LipPKS1 containing full RLs. FIG. 3B. Production levels of junction “B” RL exchanges ordered from highest KR substrate similarity with LipPKS1 (MonA2, LaidS2 and NanA2) to progressively less similarity (IdmO, AurB, SpnB) in biological triplicate.

FIG. 4A. Bimodular reductive loop exchange: Schematic of reductive loop exchanges in LipPKS2 with substrates. FIG. 4B. Phylogenetic distance, KR sequence identity, AP and MCS similarity between reductive loop donors and LipPKS. FIG. 4C. Chromatograms of RFP, LipPKS2 with donor loops SpnB and NanA2, and a structurally similar standard spiked into RFP cultures. FIG. 4D. Production levels of desired lactone in biological triplicate.

FIG. 5. Summary of results showing structures of native module and module with engineered loops, and correlation of product production with KR chemosimilarity.

FIG. 6A. Schematic of PKS processing and engineering design in this study: PKS processing of each subtype of malonyl-CoA and malonyl-CoA analog extender units. FIG. 6B. Lipomycin bimodular PKS design to produce ethyl ketones through a full reductive donor loop in LipPKS1 (blue circles), a KR mutant to abolish activity (red line), and a fused DEBS TE (red circle). FIG. 6C. Lipomycin bimodular PKS design to produce δ-lactone through an AT-swap in LipPKS1 (green circle), a full reductive donor loop in LipPKS2 (blue circles), and a fused DEBS TE (red circle).

FIG. 7A. Production of ethyl ketones and side products in engineered Lip1-Lip2 bimodular system: Schematic, MS chromatogram, and quantification of 3,5-dimethyl hexanone. FIG. 7B. Schematic, MS chromatogram and quantification of the side product 3-hydroxy-2,4-dimethylpentanoic acid due to incomplete reduction by LipPKS1. FIG. 7C. Schematic of the side product of incomplete reduction in LipPKS1 processed and elongated by LipPKS2. FIG. 7D. Schematic of the side product of complete reduction in LipPKS1 with premature hydrolysis.

FIG. 8A. Production of δ-lactone and side products in engineered Lip1-Lip2 bimodular system: Schematic, MS chromatogram, and quantification of 3-isopropyl-6-methyltetrahydropyranone. FIG. 8B. Schematic and MS chromatogram of the side product 3-hydroxy-4-methylpentanoic acid due to premature hydrolysis of LipPKS1. FIG. 8C. Schematic and MS chromatogram of the side product of incomplete reduction in LipPKS2.

FIG. 9A. Bioinformatic analysis of reductive loop exchanges: KR subtypes determine the stereochemistry of the β-hydroxyl and α-carbon. FIG. 9B. Phylogenetic tree of the ketoreductase (KR) domain of all manually curated KRs in ClusterCAD determined by ModelFinder in IQ-Tree. This evolutionary reconstruction revealed that KR-only (reductive loops with only a KR domain) B1 subtypes split from a common ancestor of fatty acid synthases and iterative PKSs. As in previous investigations, we found that KR-only B1 subtypes later resulted in the addition of DH and DH/ER domains,¹⁸ likely through recombination.¹⁹ We extend this finding to note that the KR-only B1 subtype branch diverged to produce the other KR-only subtypes (i.e. A1, A2 and B2).

FIG. 10. Structures of the final products of the recipient PKS (lipomycin) and the PKSs harboring the donor loops.

FIG. 11. Phylogenetic similarity of the native Lip1 KS domain to each donor KS, normalized to the most similar and least similar KS domain in ClusterCad. The value above each bar denotes the sequence identity percentage.

FIG. 12. Proteomics of LipPKS1 reductive loop swaps at junction A. The cells were harvested at the end of production (day 10). Three tryptic peptides that are shared in all engineered LipPKS1 were quantified using described targeted MS method. The sum peak area of these peptides was used as total protein peak area to relatively quantify across samples expressing different LipPKS1. The mean value and standard deviation of the protein peak area in three biological replicates (N=3) were plotted in the bar graph.

### DETAILED DESCRIPTION OF THE INVENTION

Unless contraindicated or noted otherwise, in these descriptions and throughout this specification, the terms “a” and “an” mean one or more, the term “or” means and/or. The examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

### Chemoinformatic-Guided Engineering of Polyketide Synthases

We sought to establish that chemoinformatics, a field traditionally used in drug discovery, offers a viable strategy for reductive loop exchanges. We first introduced a set of donor reductive loops of diverse genetic origin and chemical substrate structures into the first extension module of the lipomycin PKS (LipPKS1). Product titers of these engineered unimodular PKSs correlated with chemical similarity between the substrate of the donor reductive loops and recipient LipPKS1, reaching a titer of 165 mg/L of short chain fatty acids produced by Streptomyces albus J1074 harboring these engineered PKSs. Expanding this method to larger intermediates requiring bimodular communication, we introduced reductive loops of divergent chemosimilarity into LipPKS2 and determined triketide lactone production. Collectively, we observed a statistically significant correlation between atom pair chemosimilarity and production, establishing a new chemoinformatic method that enables the engineering of PKSs to produce desired, unnatural products.

Rational reprogramming of PKS enzymes for the biosynthesis of new polyketides has been a major research thrust over the past three decades.¹⁻³ PKSs load a malonyl-CoA analog onto the acyl carrier protein (ACP) using the acyltansferase (AT) domain and extend the growing chain from the ketosynthase (KS) domain through a decarboxylative Claisen condensation reaction. After chain extension, the β-carbonyl reduction state is determined by the module's reductive domains, namely the ketoreductase (KR), dehydratase (DH), and enoylreductase (ER), which generate the β-hydroxyl, α-β alkene, or saturated β-carbons respectively, when progressively combined. Unlike fatty acid synthases, which faithfully produce saturated fatty acids, PKSs have this variability in β-carbonyl reduction. Consequently, multiple studies have reported PKS module engineering for various β-carbon oxidation states.⁴⁻⁸ However, design strategies for introduction of reductive loop exchanges (i.e. KR-DH-ER domains) into partially reductive modules remain elusive. Here we compare bioinformatic and chemoinformatic approaches to guide reductive loop (RL) exchanges and develop a new method for RL exchanges based on the chemical similarity of the RL substrate. Chemoinformatics, an interdisciplinary field blending computational chemistry, molecular modeling and statistics to analyze structure-activity relationships, was first established for drug discovery.⁹ A chemoinformatic approach to PKS engineering could be valuable, particularly in RL exchanges where the KR and DH domains are substrate-dependent¹: acyl chain length has critically affected dehydration in stand-alone DH¹⁰ and full PKS module studies.^{7,13}

Chemoinformatic methods such as atom pair (AP) similarity, which characterizes atom pairs (e.g. length of bond path, number of π electrons), and maximum common substructure (MCS) similarity, which identifies the largest common substructure between two molecules,¹⁴ could beneficially describe substrate profiles. While divergent in chemical characterization, both similarity methods translate to a Tanimoto coefficient with a range of 0 (least similar) to 1 (most similar).¹⁴ We hypothesized that chemosimilarity between the substrates of donor and acceptor modules in RL exchanges may correlate with production levels, thereby leading to engineered modules that better control the reductive state of the β carbon.

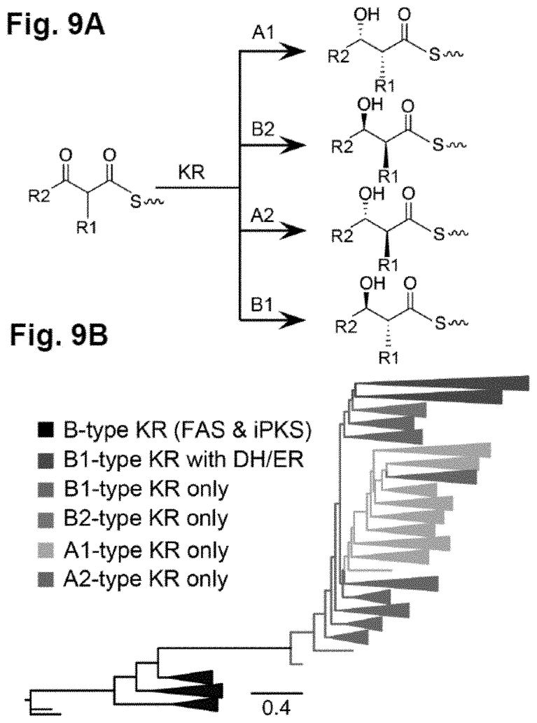

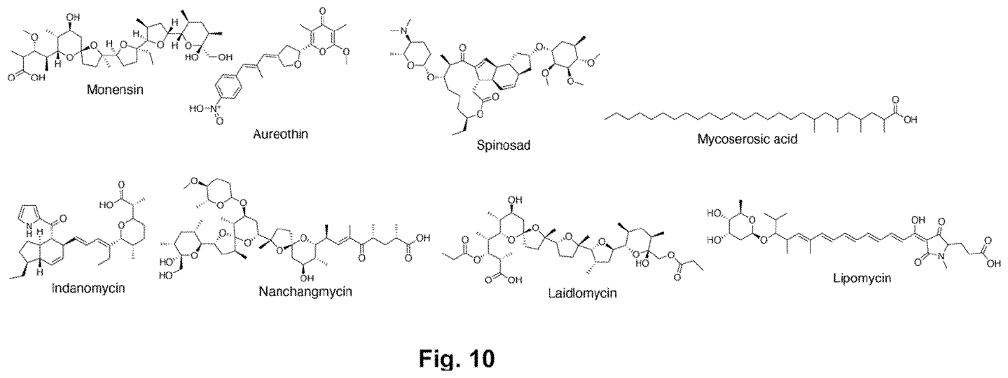

Bioinformatic studies of PKS evolution have guided engineering efforts in closely related biosynthetic gene clusters (BGCs).^{15,16} We therefore undertook a phylogenetic analysis of the reductive domain common to all RLs, the ketoreductase (KR). The KR not only reduces the β-keto group to a β-hydroxyl, but also sets the stereochemistry of the β-group and, if a branched extender is used, sets the α-carbon stereochemistry resulting in subtypes A1, A2, B 1, B2 (FIG. 9A). We generated a phylogenetic tree from all manually curated ketoreductases and ketosynthases in ClusterCAD, an online database and toolkit for Type I PKSs, totaling 72 biosynthetic gene clusters (BGCs) and 1077 modules.¹⁷ As in previous investigations,^{18,19} the KR domains clustered by subtype (FIG. 9B). In contrast, the RL type (e.g. KR, KR-DH, KR-DH-ER) did not phylogenetically cluster with its upstream or downstream KS domain (FIG. 10).¹⁸ This suggests a link between KR evolution and product specificity, analogous to the evolution of KS domains of cis-AT¹⁸ and trans-AT PKS modules^{20,21} towards substrate specificity. As KRs from KR-DH-ER modules evolved distinctly from KR-only modules, we hypothesized that neither KR sequence identity nor phylogenetic distance, a pairwise comparison of phylogenetic tree members, between the donor loops and acceptor module were likely to correlate with RL exchange production levels.

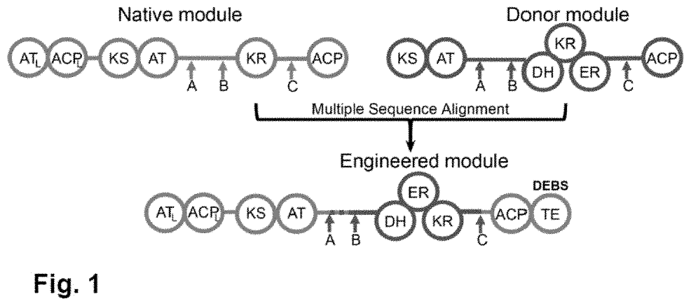

To evaluate the importance of chemical similarity and phylogenetic distance in RL exchanges, we swapped diverse, full RLs into the first module of the lipomycin PKS (LipPKS1) using conserved residues as exchange sites (FIG. 1).⁷ In our previous work, we introduced a heterologous thioesterase from 6-deoxyerythronolide B synthase (DEBS) into the C-terminus of LipPKS1; the resulting truncated PKS produced a β-hydroxy acid.²² In this work, we selected N-terminal junctions (“A” and “B”) located immediately after the post-AT linker, which is important for KS-AT domain architecture,²³ and the C-terminal junction (“C”) directly before the ACP domain (see Table S1 for sequences) based on previous work with the first module of borreledin.⁷

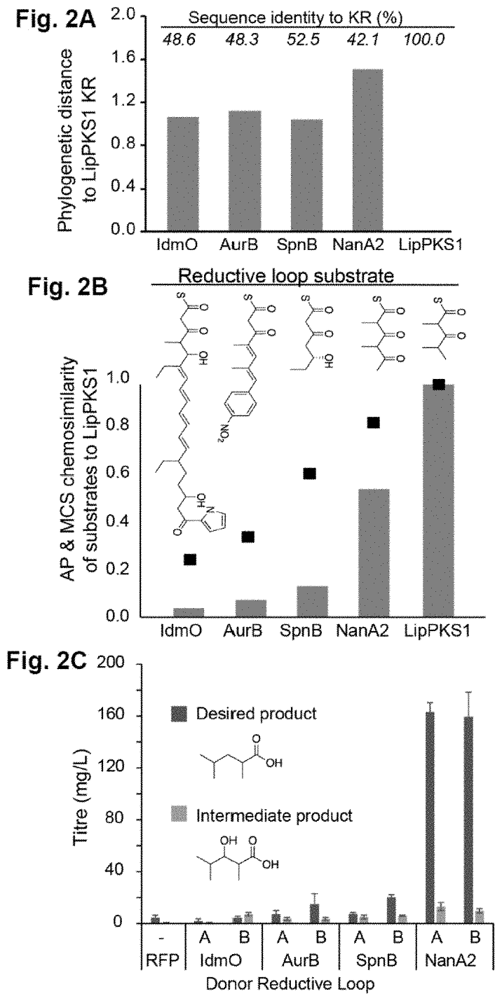

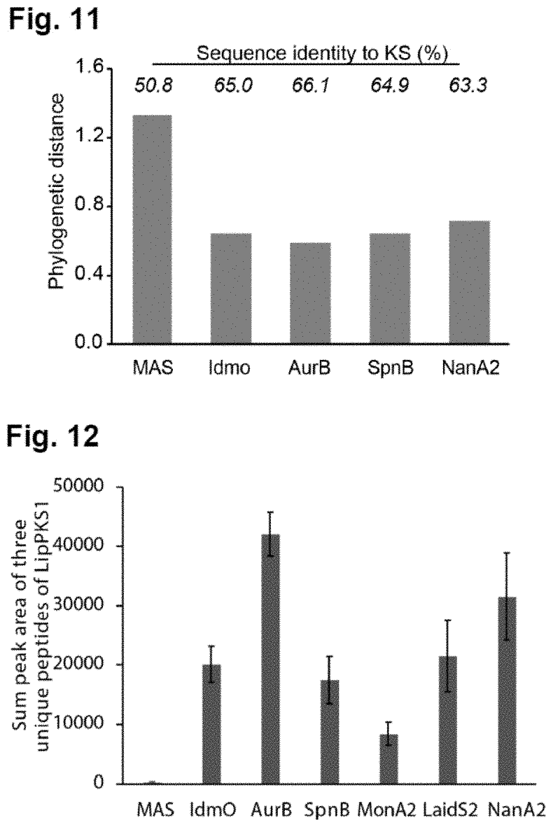

We identified four donor RLs (IdmO, indanomycin, S. antibioticus; SpnB, spinosyn, S. spinosa; AurB, aureothin, S. aureofaciens; NanA2, nanchangamycin, S. nanchangensis) to swap into LipPKS1. A pairwise comparison of phylogenetic distance and amino acid sequence identity determined that IdmO, AurB, and SpnB have the highest KR similarities to LipPKS1 (FIG. 2A). A similar trend holds in the analysis of these donor modules upstream and downstream KS domains (FIG. 11). In contrast, the NanA2 substrate has the highest chemical similarity based on AP and MCS similarity to LipPKS1, followed by SpnB (FIG. 2B). With the introduction of RL swaps, the chimeric enzymes should produce 2,4-dimethyl pentanoic acid. As in vitro PKS studies have shown divergence from in vivo results^{24,25} due to underestimation of factors including limiting substrate, crowding, and solubility,²⁶ we cloned eight chimeric modules and a control expressing red fluorescent protein (RFP), into an E. coli-Streptomyces albus shuttle vector and conjugated into S. albus J1074 (Table S1).²⁷ Following ten-day production runs in a rich medium in biological triplicate, cultures of S. albus harboring each of the constructs were harvested and analyzed for product (Supplemental Methods).

Consistent with our hypothesis, we found a perfect correlation between titers of the desired product and the AP/MCS chemosimilarities between donor and LipPKS1 module substrates (Rₛ=1.00 and p=0.00) (FIG. 2C). On the other hand, no significant correlation between product titer and phylogenetic distance or sequence similarity of the KR domain (Rₛ=0.04, p=0.60) was found. The lack of phylogenetic correlation was not surprising based on our bioinformatics analysis since the lipomycin KR is an A2-type, evolving separately from KRs with full RLs. This trend held in both junctions, though junction B chimeras generally resulted in higher product titers, consistent with a previous study of RL exchanges as the extra residues in junction A are distal to the ACP docking interface and active site.⁷ Substituting the donor loop most chemically similar to LipPKS1, NanA2, resulted in the highest titers of desired product, 2,4-dimethyl pentanoic acid, reaching 165 mg/L (Supplemental Methods). Low titers of the intermediate 2,4-dimethyl-3-hydroxypentanoic acid were produced, which we hypothesize is due to a comparatively lower rate of turnover at the energetically intensive DH domain,²⁸ allowing for premature cleavage of the stalled product by non-enzymatic or TE-mediated hydrolysis. Like our previous study of in vitro production of adipic acid, we did not detect alkene or keto acid stalled products⁷; non-functional KRs produce short chain β-keto acids that spontaneously decarboxylate to form ketones, which was also not observed, and ERs rapidly reduce trans double bonds.²⁸

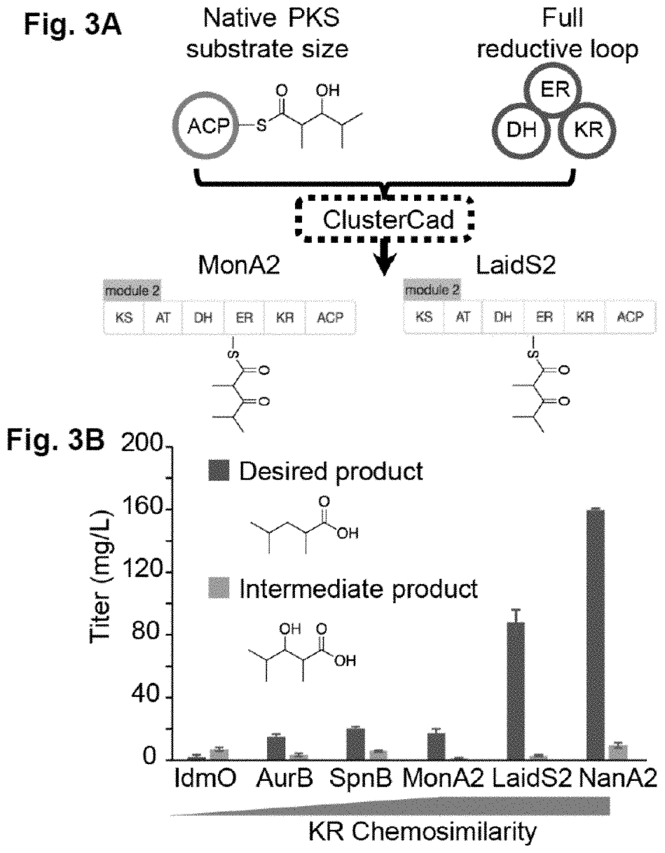

Based on these results, we took a chemoinformatic approach to further test our hypothesis that chemosimilarity of RL substrates is critical to PKS engineering. Using the ClusterCAD¹⁷ database, we identified donor RLs from laidlomycin and monensin that use a KR substrate (identical to the NanA2 KR substrate) with the highest chemically similarity to LipPKS1 (FIG. 3A). As junction B resulted in superior levels of production, the RLs of LaidS2 and MonA2 were cloned into junction B of lipomycin. Like NanA2, LaidS2 loops produced high titers of desired product, while MonA2 performed similarly to SpnB and AurB (FIG. 3B). As protein levels may influence product titers, we determined the quantitative levels of all LipPKS1 constructs using targeted proteomics at the conclusion of the production run and observed no correlation between PKS protein levels and product titers (Rₛ=−0.15 and p=0.77) (FIG. 12). Reduced protein levels in the MonA2 swap could partially explain the lower levels of production in the MonA2 swap compared to LaidS2 and NanA2. However, targeted proteomics of three peptide peaks across the PKS does not eliminate the possibility of proteolytic degradation or variability in protein quality. AP Tanimoto and MCS chemosimilarity had equivalent Spearman rank correlation to product titers (Rₛ of 0.82, p=0.045).

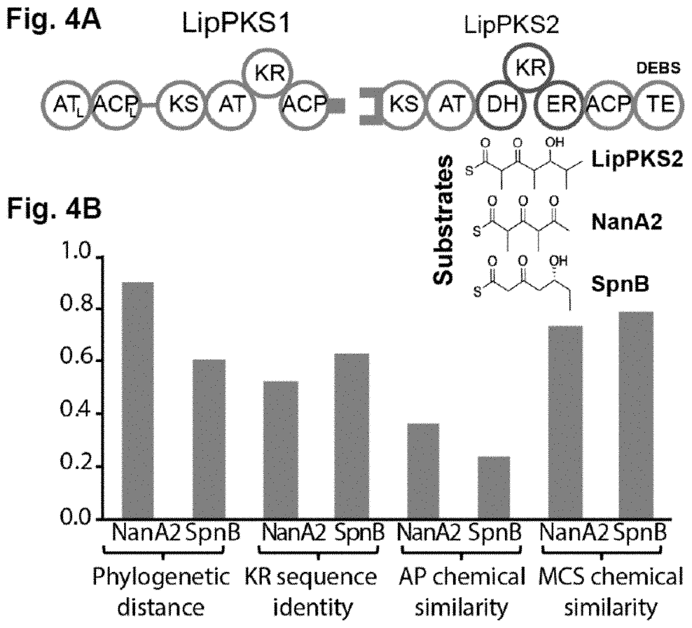

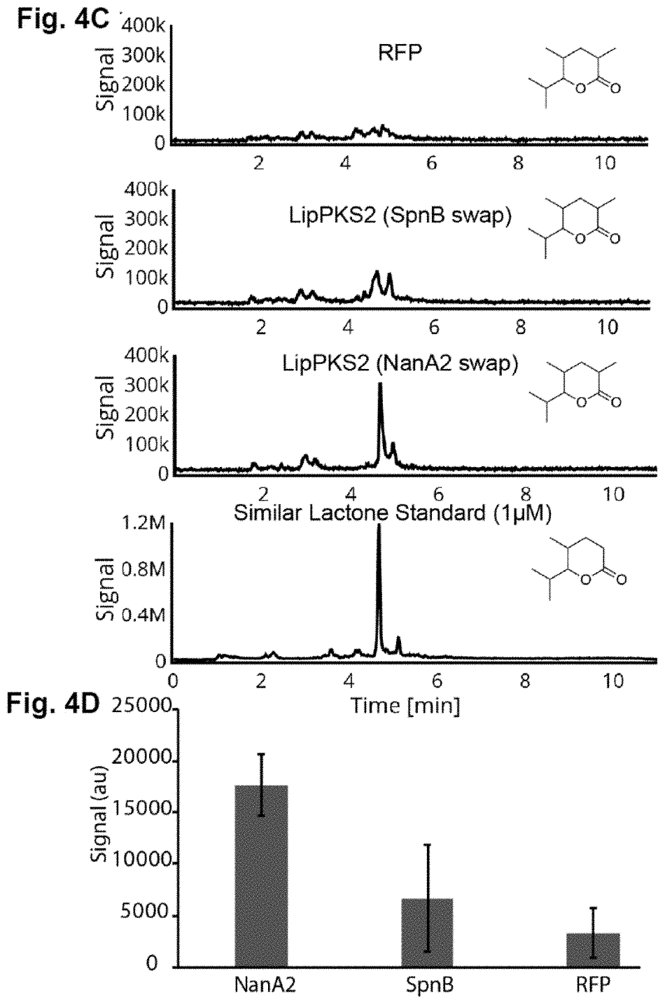

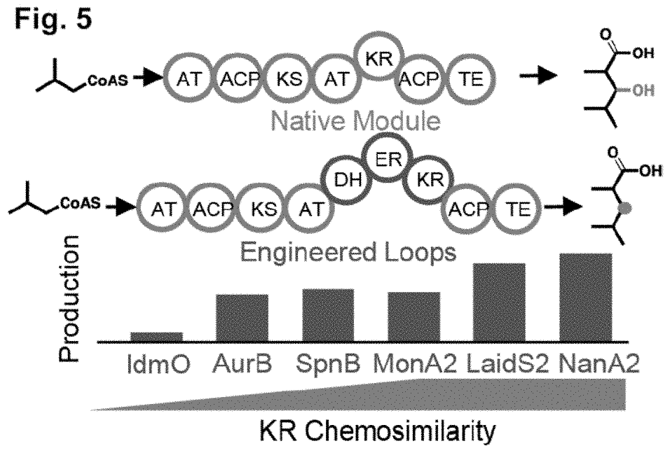

To better demonstrate the utility of this approach, we further evaluated RL exchanges where AP and MCS chemosimilarity diverge and tested this method in modules located at the center of assembly lines, thus requiring docking domain interactions and larger substrates. We therefore performed RL swaps on the second module of lipomycin, LipPKS2 (FIG. 4A), to generate triketide lactones. Donor loops from SpnB and NanA2 were selected, as NanA2 has higher AP chemosimilarity while SpnB has higher MCS chemosimilarity (FIG. 4B). As in our single-module swaps, KR phylogenetic similarity and sequence identity did not correlate with product titers. We found higher correlation with AP chemosimilarity due to higher product levels with NanA2 (FIG. 4C-D). Proteomics on each PKS of these bimodular systems was not performed to rule out the effect of variable protein levels. AP chemosimilarity more heavily weights substructures, so NanA2 and LipPKS2 have higher similarity levels because both select methylmalonyl-CoA in the first two modules. In contrast, MCS chemosimilarity simply considers the largest common substructure, which ignores the influence of commonality at the growing chain by methyl groups. This phenomenon is extendable to account for variances in chemical similarity metrics (e.g. AP, MCS), e.g. using chemosimilarity metrics that best match PKS enzymatic processing. Overall, in our reductive loop exchanges in both LipPKS1 and LipPKS2 we determined a Spearman correlation between AP Tanimoto chemosimilarity and product titer to have an Rₛ of 0.88 and a p-value of 0.004 (Supplemental Methods).

Abbreviations: PKS polyketide synthase; KS ketosynthase; AT acyltransferase; ACP acyl carrier protein; KR ketoreductase; DH dehydratase; ER enoylreductase; RL reductive loop; AP atom pair; MCS maximum common substructure; BGC biosynthetic gene cluster

### REFERENCES

(1) Barajas, J. F.; Blake-Hedges, J. M.; Bailey, C. B.; Curran, S.; Keasling, J. D. Synergy between protein and host level engineering. Synthetic and Systems Biotechnology 2017, 2, 147-166.
(2) Khosla, C.; Herschlag, D.; Cane, D. E.; Walsh, C. T. Assembly line polyketide synthases: mechanistic insights and unsolved problems. Biochemistry 2014, 53, 2875-2883.
(3) Yuzawa, S.; Zargar, A.; Pang, B.; Katz, L.; Keasling, J. D. Commodity chemicals from engineered modular type I polyketide synthases. 2018, 608, 393-415.
(4) Reid, R.; Piagentini, M.; Rodriguez, E.; Ashley, G.; Viswanathan, N.; Carney, J.; Santi, D. V.; Hutchinson, C. R.; McDaniel, R. A model of structure and catalysis for ketoreductase domains in modular polyketide synthases. Biochemistry 2003, 42, 72-79.
(5) Keatinge-Clay, A. Crystal structure of the erythromycin polyketide synthase dehydratase. J. Mol. Biol. 2008, 384, 941-953.
(6) Kellenberger, L.; Galloway, I. S.; Sauter, G.; Böhm, G.; Hanefeld, U.; Cortés, J.; Staunton, J.; Leadlay, P. F. A polylinker approach to reductive loop swaps in modular polyketide synthases. Chembiochem 2008, 9, 2740-2749.
(7) Hagen, A.; Poust, S.; Rond, T. de; Fortman, J. L.; Katz, L.; Petzold, C. J.; Keasling, J. D. Engineering a polyketide synthase for in vitro production of adipic acid. ACS Synth. Biol. 2016, 5, 21-27.
(8) Gaisser, S.; Kellenberger, L.; Kaja, A. L.; Weston, A. J.; Lill, R. E.; Wirtz, G; Kendrew, S. G.; Low, L.; Sheridan, R. M.; Wilkinson, B.; Galloway, I. S.; Stutzman-Engwall, K.; McArthur, H. A.; Staunton, J.; Leadlay, P. F. Direct production of ivermectin-like drugs after domain exchange in the avermectin polyketide synthase of Streptomyces avermitilis ATCC31272. Org. Biomol. Chem. 2003, 1, 2840-2847.
(9) Maldonado, A. G.; Doucet, J. P.; Petitjean, M.; Fan, B.-T. Molecular similarity and diversity in chemoinformatics: from theory to applications. Mol Divers 2006, 10, 39-79.
(10) Faille, A.; Gavalda, S.; Slama, N.; Lherbet, C.; Maveyraud, L.; Guillet, V.; Laval, F.; Quémard, A.; Mourey, L.; Pedelacq, J.-D. Insights into Substrate Modification by Dehydratases from Type I Polyketide Synthases. J. Mol. Biol. 2017, 429, 1554-1569.
(11) Herbst, D. A.; Jakob, R. P.; Zähringer, F.; Maier, T. Mycocerosic acid synthase exemplifies the architecture of reducing polyketide synthases. Nature 2016, 531, 533-537.
(12) Barajas, J. F.; McAndrew, R. P.; Thompson, M. G.; Backman, T. W. H.; Pang, B.; de Rond, T.; Pereira, J. H.; Benites, V. T.; Martín, H. G.; Baidoo, E. E. K.; Hillson, N. J.; Adams, P. D.; Keasling, J. D. Structural insights into dehydratase substrate selection for the borrelidin and fluvirucin polyketide synthases. J. Ind. Microbiol. Biotechnol. 2019, 46, 1225-1235.
(13) McDaniel, R.; Thamchaipenet, A.; Gustafsson, C.; Fu, H.; Betlach, M.; Ashley, G. Multiple genetic modifications of the erythromycin polyketide synthase to produce a library of novel “unnatural” natural products. Proc. Natl. Acad. Sci. USA 1999, 96, 1846-1851.
(14) Chen, X.; Reynolds, C. H. Performance of similarity measures in 2D fragment-based similarity searching: comparison of structural descriptors and similarity coefficients. J Chem Inf Comput Sci 2002, 42, 1407-1414.
(15) Peng, H.; Ishida, K.; Sugimoto, Y.; Jenke-Kodama, H.; Hertweck, C. Emulating evolutionary processes to morph aureothin-type modular polyketide synthases and associated oxygenases. Nat. Commun. 2019, 10, 3918.
(16) Awakawa, T.; Fujioka, T.; Zhang, L.; Hoshino, S.; Hu, Z.; Hashimoto, J.; Kozone, I.; Ikeda, H.; Shin-Ya, K.; Liu, W.; Abe, I. Reprogramming of the antimycin NRPS-PKS assembly lines inspired by gene evolution. Nat. Commun. 2018, 9, 3534.
(17) Eng, C. H.; Backman, T. W. H.; Bailey, C. B.; Magnan, C.; García Martín, H.; Katz, L.; Baldi, P.; Keasling, J. D. ClusterCAD: a computational platform for type I modular polyketide synthase design. Nucleic Acids Res. 2018, 46, D509—D515.
(18) Zhang, L.; Hashimoto, T.; Qin, B.; Hashimoto, J.; Kozone, I.; Kawahara, T.; Okada, M.; Awakawa, T.; Ito, T.; Asakawa, Y.; Ueki, M.; Takahashi, S.; Osada, H.; Wakimoto, T.; Ikeda, H.; Shin-Ya, K.; Abe, I. Characterization of Giant Modular PKSs Provides Insight into Genetic Mechanism for Structural Diversification of Aminopolyol Polyketides. Angew. Chem. Int. Ed. Engl. 2017, 56, 1740-1745.
(19) Jenke-Kodama, H.; Börner, T.; Dittmann, E. Natural biocombinatorics in the polyketide synthase genes of the actinobacterium Streptomyces avermitilis. PLoS Comput. Biol. 2006, 2, e132.
(20) Nguyen, T.; Ishida, K.; Jenke-Kodama, H.; Dittmann, E.; Gurgui, C.; Hochmuth, T.; Taudien, S.; Platzer, M.; Hertweck, C.; Piel, Exploiting the mosaic structure of trans-acyltransferase polyketide synthases for natural product discovery and pathway dissection. J. Nat. Biotechnol. 2008, 26, 225-233.
(21) Vander Wood, D. A.; Keatinge-Clay, A. T. The modules of trans-acyltransferase assembly lines redefined with a central acyl carrier protein. Proteins 2018, 86, 664-675.
(22) Yuzawa, S.; Eng, C. H.; Katz, L.; Keasling, J. D. Broad substrate specificity of the loading didomain of the lipomycin polyketide synthase. Biochemistry 2013, 52, 3791-3793.
(23) Tang, Y.; Kim, C.-Y.; Mathews, I. I.; Cane, D. E.; Khosla, C. The 2.7-Angstrom crystal structure of a 194-kDa homodimeric fragment of the 6-deoxyerythronolide B synthase. Proc. Natl. Acad. Sci. USA 2006, 103, 11124-11129.
(24) Khosla, C.; Tang, Y.; Chen, A. Y.; Schnarr, N. A.; Cane, D. E. Structure and mechanism of the 6-deoxyerythronolide B synthase. Annu. Rev. Biochem. 2007, 76, 195-221.
(25) Yan, J.; Hazzard, C.; Bonnett, S. A.; Reynolds, K. A. Functional modular dissection of DEBS1-TE changes triketide lactone ratios and provides insight into Acyl group loading, hydrolysis, and ACP transfer. Biochemistry 2012, 51, 9333-9341.
(26) Zotter, A.; Bäuerle, F.; Dey, D.; Kiss, V.; Schreiber, G. Quantifying enzyme activity in living cells. J. Biol. Chem. 2017, 292, 15838-15848.
(27) Phelan, R. M.; Sachs, D.; Petkiewicz, S. J.; Barajas, J. F.; Blake-Hedges, J. M.; Thompson, M. G.; Reider Apel, A.; Rasor, B. J.; Katz, L.; Keasling, Development of Next Generation Synthetic Biology Tools for Use in Streptomyces venezuelae. J. D. ACS Synth. Biol. 2017, 6, 159-166.
(28) Weber, A. L. Origin of fatty acid synthesis: thermodynamics and kinetics of reaction pathways. J. Mol. Evol. 1991, 32, 93-100.

### EXAMPLE 1

### A Bimodular PKS Platform that Expands the Biological Design Space

Traditionally engineered to produce novel pharmaceuticals, Type I modular polyketide synthases (PKSs) could be engineered as a new biosynthetic platform for the production of de novo fuels, commodity chemicals, and specialty chemicals. With their assembly-line process and vast biological design space, PKSs could be rationally programmed to sustainably produce new biomolecules with superior properties. Previously, our investigations manipulated the first module of the lipomycin PKS to produce short chain ketones, 3-hydroxy acids, and saturated, branched carboxylic acids. Building upon this work, we have expanded to multi-modular systems by engineering the first two modules of lipomycin to generate unnatural polyketides as potential biofuels and specialty chemicals in Streptomyces albus. Through reductive loop exchanges, ketoreductase knockouts, and acyltransferase swaps, we produced a novel ethyl ketone and δ-lactone as potential gasoline and fragrance specialty chemical replacements, respectively. Collectively, our work expands the biological design space to the production of “designer” biomolecules.

### Highlights

Engineered lipomycin module 1 and module 2 to produce unnatural polyketides as valuable biochemical

A reductive loop swap and ketoreductase knockout used to produce 20 mg/mL of a novel ethyl ketone, a gasoline replacement

An acyltransferase swap and reductive loop swap successfully produced δ-lactone, a potential fragrant compound.

### Introduction

Advances in biotechnology have only begun to capitalize on the biomolecular design space. Referred to as the parvome, ‘parv-’ meaning small and ‘-ome’ denoting group, the world of cell-based molecules is vastly larger than the known chemical design space (Davies, 2011). De novo biomolecular production efforts have sought to capitalize on this space to generate new biofuels, commodity chemicals, and specialty chemicals (King et al., 2016). Beyond developing molecules with superior properties, biosustainable production of these molecules could contribute to a substantial reduction in carbon emissions, which is needed to avoid potentially devastating climate change (Matthews et al., 2009). Generally, biosynthesis of unnatural molecules often relies on broad substrate ranges (Rodriguez et al., 2014) and promiscuous activity in enzymes (Khersonsky et al., 2006). While major advances have been made in protein engineering, redesigning proteins to generate novel bioactivity and achieve new products remains a major challenge (Kumar et al., 2018).

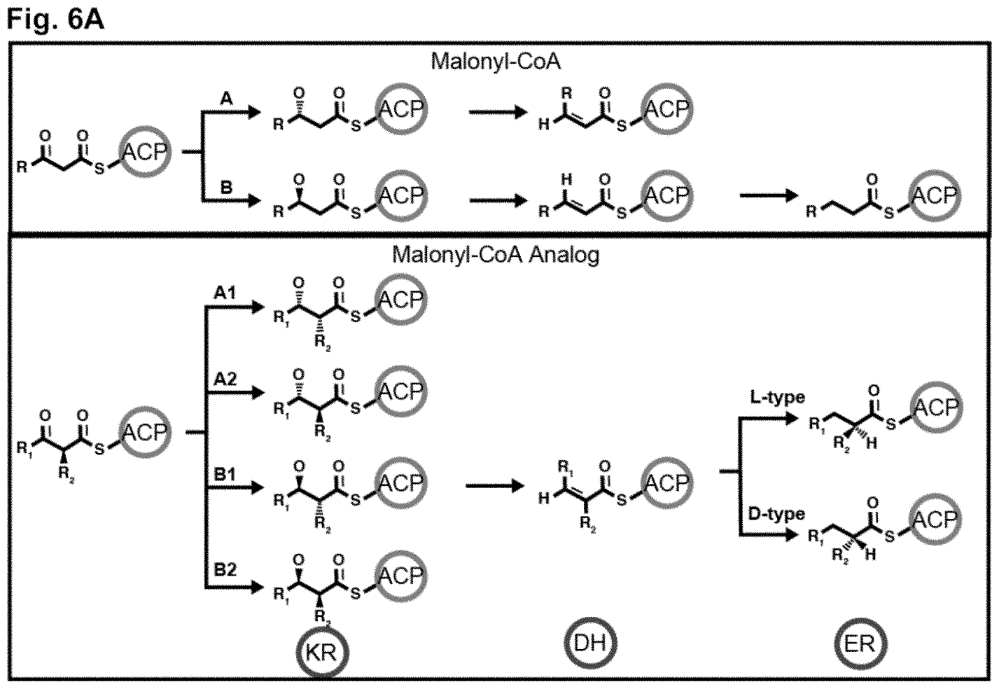

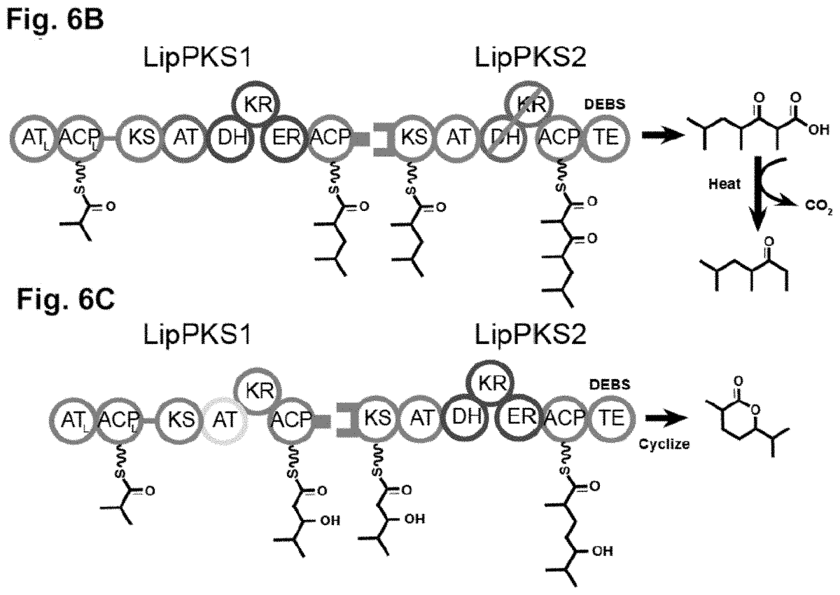

Polyketide synthases synthesize an astonishing diversity of natural products including, but not limited to, anticancer, antimicrobial, and immunomodulating compounds (reviewed by (Robbins et al., 2016)). Assembly-line-like, modular polyketide synthases (PKSs), a subset of Type I PKSs, are often linked in a collinear fashion, creating a design space that could be rationally reprogrammed to produce many valuable biomolecules (Cai and Zhang, 2018; Yuzawa et al., 2018b; Zargar et al., 2017, 2018). Each module's cycle begins with a Claisen condensation reaction between the growing chain on the ketosynthase (KS) domain and a malonyl-CoA analog on the acyl carrier protein (ACP) that was loaded by the acyltransferase domain (AT) (FIG. 6A). Unlike fatty acid synthases that exclusively incorporate malonyl-CoA, AT domains of Type-I PKSs select a wide variety of extender units, greatly expanding the biological design space. After chain extension, the molecule's carbonyl reduction state is determined by the reductive domains within a module, namely the ketoreductase (KR), dehydratase (DH), and enoylreductase (ER), which generate the β-hydroxyl, α-β alkene, or saturated (β-carbons respectively when progressively combined; PKSs have variability in (β-carbon reduction, which is a major source of polyketide diversity and another attractive feature for molecular design. Finally, a thioesterase (TE) domain typically releases the final product from the megasynthase via hydrolysis or cyclization. While these de novo pathways have most often been used to fine-tune drug candidates, combinatorial biosynthesis could be implemented to generate molecules with simple scaffolds, such as biofuels and industrial chemicals (Cai and Zhang, 2018). While combinatorial biosynthesis of PKSs through domain modification, module swaps, and other techniques have made major progress in drug development (Hertweck, 2015; Weissman, 2016; Wong and Khosla, 2012), de novo biomolecular production is still a nascent field, and there have been no examples of multi-modular PKS redesign to produce valuable biochemicals.

Previously, our group has engineered three major PKS elements in the first module of lipomycin: 1) an inserted TE to produce 3-hydroxy acids (Yuzawa et al., 2017a), 2) a KR knockout and AT domain swap to produce short chain ketones (Yuzawa et al., 2018a, 2017b), and 3) reductive loop (RL) exchanges to produce saturated, short chain carboxylic acids (Zargar et al., 2019). The design space expands considerably with multiple module systems, and in this work, we build on our single module platform by combining multiple PKS manipulations (KR knockouts, RL swaps, AT swaps, fused TE) in a biomodular system to produce novel biomolecules, namely biofuels and specialty chemicals.

### Production of Ethyl Ketones

Short chain ketones (C3-C7) have been noted for their potential as gasoline additives because of their high octane numbers (McCormick et al., 2017), and we recently tested their fuel combustion properties in a gasoline called CARBOB, a specially formulated Blendstock for Oxygenate Blending formula mandated by the state of California (Yuzawa et al., 2018a). While most ketones showed superior properties to the common biofuel butanol, methyl-branched C5 and C6 ketones even had comparable fuel properties to isooctane (octane numbers, energy density, boiling point, melting point, and flash point). Longer chain ketones (above C7) were too expensive to synthesize for testing, but it is likely they are candidates as gasoline blending agents as well, and possibly possess combustion properties comparable or superior to traditional gasoline molecules. We therefore sought to produce longer chain ketones as an example of de novo production of biomolecules in a bimodular system.

As illustrated in FIG. 6B, we aimed to produce ethyl ketones through an RL exchange in LipPKS1 and an AT swap in LipPKS2. Previously, we had performed an RL exchange in LipPKS1 with a fused DEBS TE, and found a correlation between successful production of the desired product and the chemical similarity of the donor and recipient reductive loops with the most production through the chimera of LipPKS1 with an inserted donor loop form NanA2 (nanchangamycin, module 2) (Zargar et al., 2019). In the work presented here, we replaced the fused DEBS (6-deoxyerythronolide) thioesterase with the native docking domain of LipPKS1. For Li p PKS2, we synthesized the codon-optimized gene with the native docking domain and a single point mutation at S1547A to mutate the catalytic serine to alanine. With the thioesterase of DEBS inserted following the ACP domain of LipPKS2, the programmed product of engineered Lip1-Lip2 is a β-keto carboxylic acid, which, upon acidification and heat, is an ethyl ketone, 3,5-dimethyl hexanone.

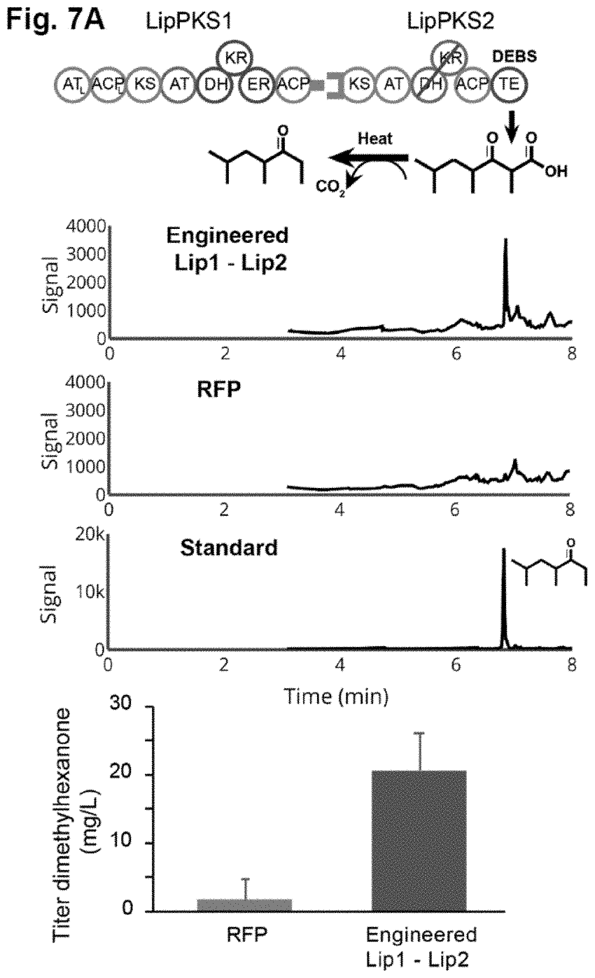

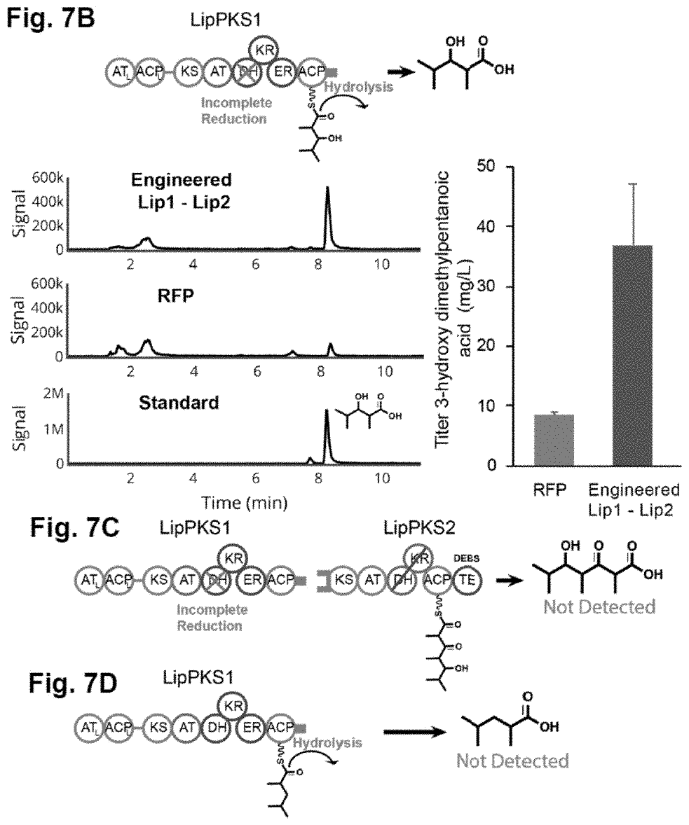

To produce the ethyl ketones, we conjugated the engineered Lip1 and Lip2 with the phiC31 and VWB integrases, respectively, into the genome of Streptomyces albus J1074. After 10-day production runs, we harvested the samples and measured titers of the final product and side products. We observe production of the desired product after heating and acidification with a titer of 20.6 mg/L (FIG. 7A). While successful, this was a considerable drop in titer compared to the 165 mg/L of the saturated, carboxylic acid produced by the singular Lip1 extension module with NanA2 reductive loops and a fused TE. This loss of production is partially reflected in the amount of side products generated in the bimodular system. Previously, we found that the unimodular LipPKS with NanA2 reductive loops and a fused TE produced the DH-stalled product, 3-hydroxy dimethyl pentanoic acid (Zargar et al., 2019). We therefore suspected that incomplete (β-carbon reduction by the engineered module 1 could cause premature hydrolysis of the product off the Lip1 ACP, resulting in production of the DH-stalled product, 3-hydroxy 2,4-dimethyl pentanoic acid, which was produced at a titer of 40 mg/L, considerably higher than that produced by the negative control (FIG. 7B). Previous studies on cis-AT PKS modules have shown that elongation by the Claisen condensation reaction has higher selectivity than acylation of the KS, a process generally known as gatekeeping (Watanabe et al., 2003; Wu et al., 2004). This may also explain the loss in yield compared to the engineered unimodular Lip1 (˜165 mg/L), as the stalled KS could reduce turnover. However, we did not observe this stalled β-hydroxy compound passed onto LipPKS2 and processed by the second module (FIG. 7C). Importantly, the native Lip1 KR and donor NanA2 RL loop produce β-hydroxy compounds with different stereochemistries (A2 type compared to B 1, FIG. 6A). As it has been shown that KR domain exchanges generally retain native stereospecificity (Kao et al. 1998), we hypothesize that this difference in stereochemistry likely causes the downstream Lip2 KS to fail to elongate the stalled B1 type β-hydroxy substrate of the engineered LipPKS1. On the other hand, we did not detect any of the fully reduced product, 2,4-dimethyl pentanoic acid, prematurely hydrolyzing as a saturated acid (FIG. 7D). This is keeping with other studies that show KS domains have less promiscuity with bulkier substrates (Jenner, 2016).

### Production of δ-Lactones

Over the past ten years, the market for genetically modified microbial production of fragrance aroma chemicals has grown, and an important fragrant compound class is δ-lactones (Gupta, 2015). While not commercially used, 3-isopropyl-6-methyltetrahydropyranone has been synthesized previously as a potentially fragrant δ-lactone (Plessis and Derrer, 2001). The engineered production of this compound requires a combination of an AT swap in LipPKS1 and a RL swap in LipPKS2, which has not been shown before. Previously, we analyzed AT domains and associated linkers to identify the boundaries for AT swaps while maintaining enzyme activity in LipPKS1 (Yuzawa et al., 2017b). An exchange of the native methylmalonyl-CoA of LipPKS1 with malonyl-CoA of the first module of the borreledin PKS (BorAT1) with a fused TE produced 3-hydroxy carboxylic acids, which could be used as organic building blocks (Yuzawa et al., 2017b). Here, we replaced the fused TE with the native Lip1 docking domains. For LipPKS2, we had previously introduced the NanA2 reducing loop, and when combined with the native LipPKS1, we engineered production of 3-isopropyl-4,6-dimethyltetrahydropyranone (Zargar et al., 2019). This combination of LipPKS1 with the AT swap and LipPKS2 with an RL exchange should produce 3-isopropyl-6-methyltetrahydropyranone (FIG. 2A).

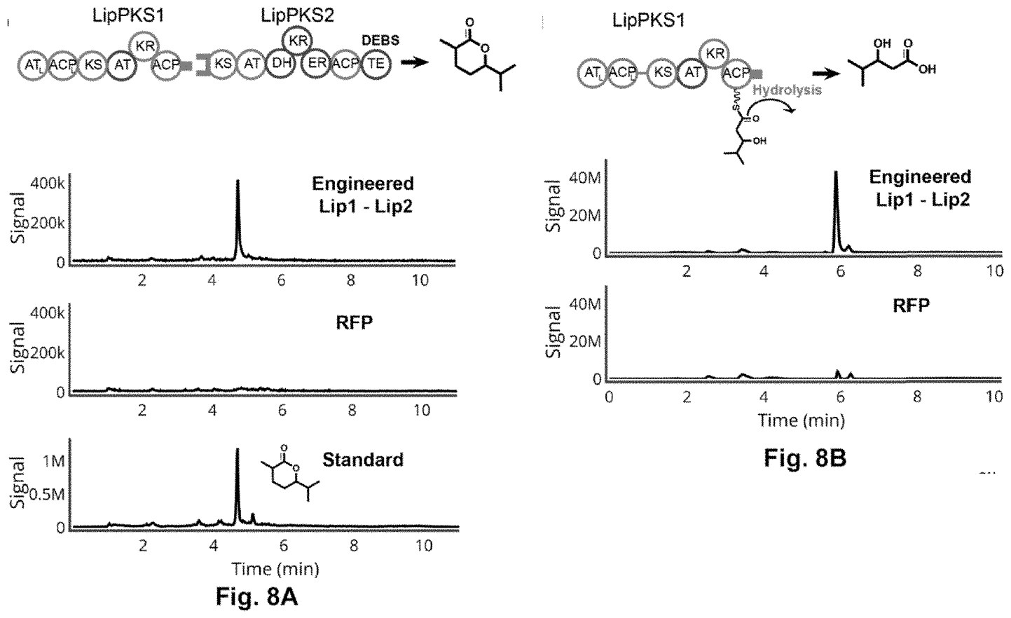

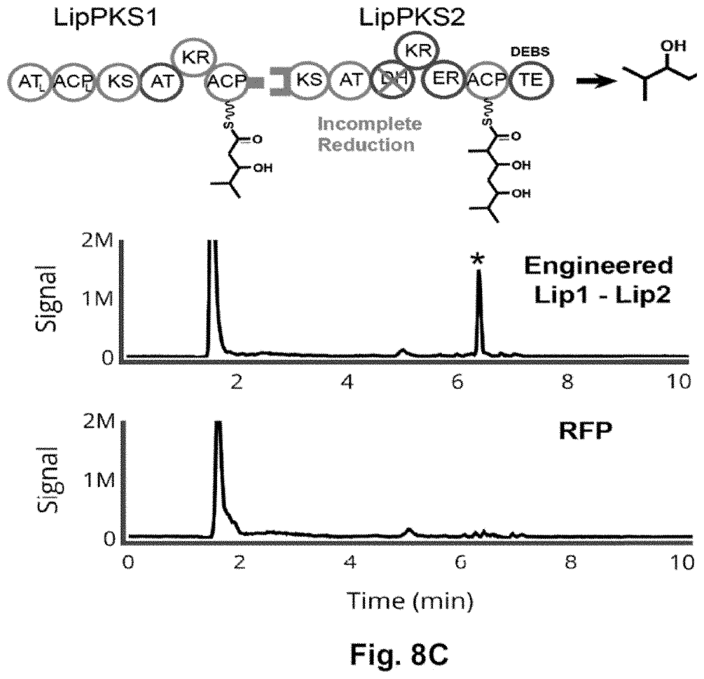

As before, we integrated the genes encoding the engineered LipPKS1 and LipPKS2 into S. albus J1074 at the phiC31 and VWB phage attachment sites with phiC31 and VWB integrases, respectively. After 10 day production runs, we detected the programmed product with a titer of 40 μg/L (FIG. 8A). This yield is considerably lower than the ethyl ketone titers from a similar bimodular PKS (20 mg/L). Manipulation of the first module resulted in side products generated by premature hydrolysis at the LipPKS1. The AT swapped LipPKS1 produced 3-hydroxy 4-methyl pentanoic acid, and we found peaks of thet mass correlated to the premature hydrolysis product (FIG. 8B). As expected, we also observed production of 3,5-dihydroxy 2,4-dimethyl heptanoic acid, as the KR domain of the NanA2 reductive loop within LipPKS2 did not fully dehydrate the intermediate (FIG. 8C). Intermediate products in the cyclized form were not detected.

### Discussion

While unaltered natural products and synthetic chemistry have been the basis of industrial molecules, de novo biomolecular designs contain an astonishing diversity of molecules that could have a transformational impact in many fields (Smanski et al., 2016). A biosynthetic platform based on PKSs represents a vast design space with an attractive programming basis. With the diversity of starter substrates (˜10²), malonyl-CoA analogs (˜10¹), and stereochemistry arrangements (˜10¹), a unimodular system alone can feasibly produce over 10,000 molecules and each subsequent module increases the number by two orders of magnitude. Integrating other fields such as ‘click’ chemistry obtains new capabilities (Le Feuvre and Scrutton 2018; Kalkreuter et al. 2019; Zhu et al. 2015).

Previously, we engineered the first module of lipomycin to make an array of molecules through AT swaps, RL swaps, KR knockouts, and a fused TE. Here, we have shown that we can leverage the knowledge we gained in unimodular systems to combine engineering manipulations, and successfully generate novel biofuels and specialty chemicals in the host Streptomyces albus.

### Materials and Methods: Cloning of All Constructs

All clusters were expressed from the S. albus chromosome under control of the GapDH(E1) promoter from Eggerthella lenta. Junction sites for reductive loop exchanges were determined by those reported by Hagen et al. through multiple sequence alignment with MUSCLE (Hagen et al. 2016; Edgar 2004). The plasmids along with their associated information have been deposited in the public version of JBEI registry.

Cloning of LipPKS1 with Full Reductive Loop Modules and Native Docking Domain

The phiC31 Streptomyces integrase vectors were used as described by Phelan et al. to integrate the LipPKS1 reductive loop swap modules (Phelan et al., 2017). The native docking domain sequences of LipPKS1 were codon optimized for E. coli and synthesized by Gen9 (since acquired by Ginkgo Bioworks). They were cloned through Golden Gate assembly into the LipPKS1 module with an inserted RL from NanA2 from Zargar et al (Zargar et al., 2019).

Cloning of LipPKS1 with AT-Swap and Native Docking Domain

The phiC31 integrase vectors (Phelan et al., 2017) were used to integrate the AT-swapped LipPKS1 module into the phiC31 site in the S. albus chromosome. The Lip1 native docking domain sequences of LipPKS1 were cloned into the AT-swapped LipPKS1 module from Yuzawa et al. (Yuzawa et al., 2017b) through Golden Gate assembly.

Cloning of LipPKS2 with KR Knockout and Fused DEBS Thioesterase

The VWB Streptomyces integrase vectors were used to integrate the LipPKS2 KR⁻ module (Phelan et al., 2017). The native LipPKS2 was codon optimized for E. coli with a single point mutation S1547A into the KR active site to mutate the catalytic serine to alanine, thereby abolishing KR activity, and synthesized by Gen9 (since acquired by Ginkgo Bioworks). The fused DEBS thioesterase domain was placed at the C-terminus of the ACP domain through Golden Gate assembly.

Cloning of LipPKS2 with Full Reductive Loop Modules and Fused DEBS Thioesterase

The VWB Streptomyces integrase vectors were used to integrate the LipPKS2 reductive loop modules cloned previously (Zargar et al., 2019).

Conjugation of phiC31 Integrase LipPKS1 Constructs into Streptomyces albus J1074

E. coli ET12567/pUZ8002 was transformed with LipPKS1 plasmids and selected for on LB agar containing kanamycin (25 μg/mL), chloramphenicol (15 μg/mL), and apramycin (50 μg/mL). A single colony was inoculated into 5 mL of LB containing kanamycin (25 μg/mL), chloramphenicol (15 μg/mL), and apramycin (50 μg/mL) at 37° C. The overnight culture was used to seed 10 mL of LB containing the same antibiotics, and the new culture was grown at 37° C. to an OD600 of 0.4-0.6. The E. coli cells were pelleted by centrifugation, washed twice with LB, and resuspended in 500 μL of LB. Fresh S. albus J1074 spores were collected from a mannitol soy agar plate with 5 mL of 2×YT and incubated at 50° C. for 10 min. The spores (500 μL) and the E. coli cells (500 μL) were mixed, spread onto mannitol soy agar, and incubated at 30° C. for 16 hours. 1 mL of both nalidixic acid (20 μg/mL) and apramycin (40 μg/mL) were added to the plate and allowed to dry. The plate was then incubated for 3-4 days at 30° C. A single colony was inoculated into TSB containing nalidixic acid (25 μg/mL) and apramycin (25 μg/mL). After 3-4 days, a 1 mL aliquot was taken for genomic isolation using the Maxwell kit (Promega, Cat# AS1490, Madison Wis.). Successful integration was verified using qPCR. The remainder of the culture was spread onto a MS plate and incubated at 30° C. for 2-3 days. The spores were collected from the plate with 3-4 mL of water and mixed with glycerol to prepare a 25% glycerol stock, which was stored at −80° C. for long-term storage.

Conjugation of VWB Integrase LipPKS2 Constructs into Streptomyces albus

E. coli ET12567/pUZ8002 was transformed with LipPKS2 plasmids and selected for on LB agar containing kanamycin (25 μg/mL), chloramphenicol (15 μg/mL), and spectinomycin (200 μg/mL). A single colony was inoculated into 5 mL of LB containing kanamycin (25 μg/mL), chloramphenicol (15 μg/mL), and spectinomycin (200 μg/mL) at 37° C. The overnight culture was used to seed 10 mL of LB containing the same antibiotics, and the new culture was grown at 37° C. to an OD600 of 0.4-0.6. The E. coli cells were pelleted by centrifugation, washed twice with LB, and resuspended in 500 μL of LB. S. albus J1074 spores with an integrated LipPKS1, which were collected from a mannitol soy agar plate with 5 mL of 2×YT and incubated at 50° C. for 10 min. The spores (500 μL) and the E. coli cells (500 μL) were mixed, spread onto mannitol soy agar, and incubated at 30° C. for 16 hours. 1 mL of each nalidixic acid (20 μg/mL), apramycin (40 μg/mL), and spectinomycin (400 μg/mL) was added to the plate and allowed to dry. The plate was then incubated for 3-4 days at 30° C. A single colony was inoculated into TSB containing nalidixic acid (25 μg/mL), apramycin (25 μg/mL) and spectinomycin (200 μg/mL). After 3-4 days, a 1 mL aliquot was taken for genomic isolation through the Maxwell kit (Promega, Cat# AS1490, Madison Wis.). Successful integration was verified through qPCR. The remainder of the culture was spread onto a MS plate and incubated at 30° C. for 2-3 days. The spores were collected from the plate with 3-4 mL of water and mixed with glycerol to prepare a 25% glycerol stock, which was stored at -80° C. for long-term storage.

S. albus Production Runs

Engineered S. albus spores were grown in 12 mL of TSB medium containing nalidixic acid (50 μL), apramycin (50 μg/mL) and spectinomycin (200 μg/mL) for 4-5 days at 30° C. Three mL of the overnight culture was used to seed 30 mL of 10% media 042 and 90% plant hydrolysate (Yuzawa et al., 2018a), supplemented with 2.4 grams/liter of valine and nalidixic acid (50 μg/mL), which was grown for 10 days at 30° C. For production runs of lactones, an overlay of 4 mL of dodecane was added to retain the product.

### Sample Preparation for Detection of Acids

To detect acid side products, 1 mL of each sample was centrifuged at 5000 g for 10 minutes and 200 μL of the supernatant was removed. The supernatant was mixed with 200 μl of 100 μM hexanoic acid dissolved in methanol and filtered using Amicon Ultra Centrifugal filters, 3 KDa Ultracel, 0.5 mL device (Millipore). β-hydroxy (3-hydroxy-2,4-dimethylpentanoic acid) and saturated acids (2,4-dimethylpentanoic acid) were synthesized by Enamine (Cincinnati, USA) to greater than 95% purity.

### Sample Preparation for Bimodular Production of Triketide Lactones

10 mL of each sample was mixed with 2 mL of diethyl ether in a 15-mL conical tube and vortexed for 5 minutes. Each conical tube was centrifuged at 5000 g for 10 minutes and 1 mL of ether was removed and placed into a 2-mL flat bottom microcentrifuge tube. Air was gently blown over each sample in a chemical fume hood until dry. The extract was resuspended in 200 μL of methanol. 5-methyl-6-(propan-2-yl)oxan-2-one was synthesized by Enamine (Cincinnati, USA) to greater than 95% purity.

### Sample Preparation for Detection of 3,5-Dimethyl Hexanone

One mL of each sample was harvested in a 1.7-mL microcentrifuge tube. To each tube, 300 μL of ethyl acetate and 50 μL uL of formic acid were added. All tubes were wrapped in paraffin and heated for 60 minutes at 80° C. Samples were then placed on ice for 5 minutes and vortexed for 5 minutes. Each sample was centrifuged for 2 minutes at 10,000 g. One hundred microliters of ethyl acetate was removed from each sample and placed in a GC MS vial.

### Analytical Chemistry

### GC-MS Detection of 3,5-Dimethyl Hexanone

Electron ionization GC/MS analysis was performed on a G3950A-9000 GC (Agilent) using a J&W HP-5 ms Ultra Inert Intuvo GC column module (15 m length, 0.25 mm inner diameter, 0.25 μm film thickness). The GC was coupled to a mass selective detector (Agilent 5977B MSD) and an autosampler (Model 7693 Agilent). The GC oven was programmed at 60° C. for 3 minutes, ramping at 10° C./min until 120° C., and then ramping at 200° C./min to 300° C.; the injection port temperature was 250° C. Using an authentic standard, we determined a single-ion method of detection collecting data at m/z=57.00, m/z=85.00, m/z=142.00.

### LC-MS Detection of Short Chain Acids

LC separation of short chain acids was conducted on an InfinityLab Poroshell HPHB-C18 reversed phase column (100 mm length, 3.0 mm internal diameter, 2.7 μm particle size; Agilent, United States) using a Waters Acquity Autopurification system, prep UHPLC-MS (ESI) (Waters, United States). The mobile phase for separating 2,4-dimethylpentanoic acid and 2,4-dimethylpent-2-enoic acid was composed of 10 mM ammonium acetate and 0.05% ammonium hydroxide in water (solvent A) and 10 mM ammonium acetate and 0.05% ammonium hydroxide in methanol (solvent B). The mobile phase for separating 3-hydroxy-2,4-dimethylpentanoic acid and 2,3-dimethyl-3-oxopentanoic acid was composed of 0.1% formic acid in water (solvent A) and 0.1% formic acid in methanol (solvent B). All acids were each separated via the following gradient: increased from 5 to 97.1% B in 6.5 min, held at 97.1% B for 1.3 min, decreased from 97.1 to 5% B in 0.4 min, and held at 5% B for an additional 2 min. The flow rate was held at 0.42 ml·min−1 for 8.2 min, and then increased from 0.42 to 0.65 ml·min−1 for an additional 2 min. The total LC run time was 10.8 min. Samples of 3 μl were injected into the LC column. Acids were detected via [M−H]-ions. The Agilent 1200 Rapid Resolution LC system was coupled to an Agilent 6210 TOF (Agilent Technologies, United States). Nitrogen gas was used as both the nebulizing and drying gas to facilitate the production of gas-phase ions. The drying and nebulizing gases were set to 111·min⁻¹ and 301·bin⁻², respectively, and a drying gas temperature of 330° C. was used throughout. Atmospheric pressure chemical ionization was conducted in the positive-ion mode with capillary and fragmentor voltages of 3.5 kV and 100 V, respectively. The skimmer, OCT1 RF, and corona needle were set to 50 V, 170 V, and 4 μA, respectively. The vaporizer was set to 350° C. The analysis was performed using an m/z range of 70 to 1100. Data acquisition and processing were performed using MassHunter software (Agilent Technologies, United States).

### LC-MS Detection of Triketide Lactones

LC separation of triketide lactones was conducted on a Kinetex XB-C18 reversed phase column (100 mm length, 3 mm internal diameter, 2.6 μm particle size; Phenomenex, United States) using an Agilent 1200 Rapid Resolution LC system (Agilent Technologies, United States). The mobile phase was composed of water (solvent A) and methanol (solvent B). Lactones were each separated via the following gradient: increased from 30 to 90% B in 3.7 min, held at 94% B for 5.2 min, decreased from 90 to 30% B in 0.33 min, and held at 30% B for an additional 2.0 min. The flow rate was held at 0.42 ml·min⁻¹ for 8.67 min, increased from 0.42 to 0.60 ml·min⁻¹ in 0.33 min, and held at 0.60 ml·min⁻¹ for an additional 2.0 min. The total LC run time was 11.0 min. The column compartment and autosampler temperatures were set to 50° C. and 6° C., respectively. Samples of 3 μl were injected into the LC column. The Agilent 1200 Rapid Resolution LC system was coupled to an Agilent 6210 TOF (Agilent Technologies, United States). Nitrogen gas was used as both the nebulizing and drying gas to facilitate the production of gas-phase ions. The drying and nebulizing gases were set to 101·min⁻¹ and 251·bin⁻², respectively, and a drying gas temperature of 325° C. was used throughout. Atmospheric pressure chemical ionization was conducted in the positive-ion mode with capillary and fragmentor voltages of 3.5 kV and 100 V, respectively. The skimmer, OCT1 RF, and corona needle were set to 50 V, 170 V, and 4 μA, respectively. The vaporizer was set to 350° C. Lactones were detected via [M+H]⁺ ions: m/z=157.1223. The analysis was performed using an m/z range of 70 to 1100. Data acquisition and processing were performed using MassHunter software (Agilent Technologies, United States).

### BIBLIOGRAPHY

Cai, W., Zhang, W., 2018. Engineering modular polyketide synthases for production of biofuels and industrial chemicals. Curr. Opin. Biotechnol. 50, 32-38. doi:10.1016/j.copbio.2017.08.017
Davies, J., 2011. How to discover new antibiotics: harvesting the parvome. Curr. Opin. Chem. Biol. 15, 5-10. doi:10.1016/j.cbpa.2010.11.001
Eng, C. H., Backman, T. W. H., Bailey, C. B., Magnan, C., Garcia Martin, H., Katz, L., Baldi, P., Keasling, J. D., 2018. ClusterCAD: a computational platform for type I modular polyketide synthase design. Nucleic Acids Res. 46, D509—D515. doi:10.1093/nar/gkx893
Gupta, C., 2015. A biotechnological approach to microbial based perfumes and flavours. JMEN 2. doi:10.15406/jmen.2015.02.00034
Hertweck, C., 2015. Decoding and reprogramming complex polyketide assembly lines: prospects for synthetic biology. Trends Biochem. Sci. 40, 189-199. doi:10.1016/j.tibs.2015.02.001
Jenner, M., 2016. Substrate Specificity of Ketosynthase Domains Part III: Elongation-Based Substrate Specificity, in: Using Mass Spectrometry for Biochemical Studies on Enzymatic Domains from Polyketide Synthases, Springer Theses. Springer International Publishing, Cham, pp. 131-154. doi:10.1007/978-3-319-32723-5_6
Jenner, M., Afonso, J. P., Bailey, H. R., Frank, S., Kampa, A., Piel, J., Oldham, N. J., 2015. Acyl-chain elongation drives ketosynthase substrate selectivity in trans-acyltransferase polyketide synthases. Angew Chem Int Ed Engl 54, 1817-1821. doi:10.1002/anie.201410219
Kalkreuter, E., CroweTipton, J. M., Lowell, A. N., Sherman, D. H., Williams, G. J., 2019. Engineering the Substrate Specificity of a Modular Polyketide Synthase for Installation of Consecutive Non-Natural Extender Units. J. Am. Chem. Soc. 141, 1961-1969. doi:10.1021/jacs.8b10521
Khersonsky, O., Roodveldt, C., Tawfik, D. S., 2006. Enzyme promiscuity: evolutionary and mechanistic aspects. Curr. Opin. Chem. Biol. 10, 498-508. doi:10.1016/j.cbpa.2006.08.011
King, J. R., Edgar, S., Qiao, K., Stephanopoulos, G., 2016. Accessing Nature's diversity through metabolic engineering and synthetic biology. [version 1; peer review: 2 approved]. F1000Res. 5. doi:10.12688/f1000research.7311.1
Kumar, A., Wang, L., Ng, C. Y., Maranas, C. D., 2018. Pathway design using de novo steps through uncharted biochemical spaces. Nat. Commun. 9, 184. doi:10.1038/s41467-017-02362-x
Le Feuvre, R. A., Scrutton, N. S., 2018. A living foundry for Synthetic Biological Materials: A synthetic biology roadmap to new advanced materials. Synthetic and Systems Biotechnology 3, 105-112. doi:10.1016/j.synbio.2018.04.002
Matthews, H. D., Gillett, N. P., Stott, P. A., Zickfeld, K., 2009. The proportionality of global warming to cumulative carbon emissions. Nature 459, 829-832. doi:10.1038/nature08047
McCormick, R. L., Fioroni, G., Fouts, L., Christensen, E., Yanowitz, J., Polikarpov, E., Albrecht, K., Gaspar, D. J., Gladden, J., George, A., 2017. Selection Criteria and Screening of Potential Biomass-Derived Streams as Fuel Blendstocks for Advanced Spark-Ignition Engines. SAE Int. J. Fuels Lubr. 10, 442-460. doi:10.4271/2017-01-0868
Phelan, R. M., et al., 2017. Development of Next Generation Synthetic Biology Tools for Use in Streptomyces venezuelae. ACS Synth. Biol. 6, 159-166. doi:10.1021/acssynbio.6b00202
Plessis, C., Derrer, S., 2001. Novel photolactonisation from xanthenoic esters. Tetrahedron Lett. 42, 6519-6522. doi:10.1016/50040-4039(01)01296-5
Robbins, T., Liu, Y.-C., Cane, D. E., Khosla, C., 2016. Structure and mechanism of assembly line polyketide synthases. Curr. Opin. Struct. Biol. 41, 10-18. doi:10.1016/j.sbi.2016.05.009
Rodriguez, G. M., Tashiro, Y., Atsumi, S., 2014. Expanding ester biosynthesis in Escherichia coli. Nat. Chem. Biol. 10, 259-265. doi:10.1038/nchembio.1476
Smanski, M. J., Zhou, H., Claesen, J., Shen, B., Fischbach, M. A., Voigt, C. A., 2016. Synthetic biology to access and expand nature's chemical diversity. Nat. Rev. Microbiol. 14, 135-149. doi:10.1038/nrmicro.2015.24
Watanabe, K., Wang, C. C. C., Boddy, C. N., Cane, D. E., Khosla, C., 2003. Understanding substrate specificity of polyketide synthase modules by generating hybrid multimodular synthases. J. Biol. Chem. 278, 42020-42026. doi:10.1074/jbc.M305339200
Weissman, K. J., 2016. Genetic engineering of modular PKSs: from combinatorial biosynthesis to synthetic biology. Nat. Prod. Rep. 33, 203-230. doi:10.1039/c5np00109a
Wong, F. T., Khosla, C., 2012. Combinatorial biosynthesis of polyketides—a perspective. Curr. Opin. Chem. Biol. 16, 117-123. doi:10.1016/j.cbpa.2012.01.018
Wu, J., Kinoshita, K., Khosla, C., Cane, D. E., 2004. Biochemical analysis of the substrate specificity of the beta-ketoacyl-acyl carrier protein synthase domain of module 2 of the erythromycin polyketide synthase. Biochemistry 43, 16301-16310. doi:10.1021/bi048147g
Yuzawa, S., Bailey, C. B., Fujii, T., Jocic, R., Barajas, J. F., Benites, V. T., Baidoo, E. E. K., Chen, Y., Petzold, C. J., Katz, L., Keasling, J. D., 2017a. Heterologous Gene Expression of N-Terminally Truncated Variants of LipPks1 Suggests a Functionally Critical Structural Motif in the N-terminus of Modular Polyketide Synthase. ACS Chem. Biol. 12, 2725-2729. doi:10.1021/acschembio.7b00714
Yuzawa, S., Deng, K., Wang, G., Baidoo, E. E. K., Northen, T. R., Adams, P. D., Katz, L., Keasling, J. D., 2017b. Comprehensive in Vitro Analysis of Acyltransferase Domain Exchanges in Modular Polyketide Synthases and Its Application for Short-Chain Ketone Production. ACS Synth. Biol. 6, 139-147. doi:10.1021/acssynbio.6b00176
Yuzawa, S., et al, 2018a. Short-chain ketone production by engineered polyketide synthases in Streptomyces albus. Nat. Commun. 9, 4569. doi:10.1038/s41467-018-07040-0
Yuzawa, S., Zargar, A., Pang, B., Katz, L., Keasling, J. D., 2018b. Commodity chemicals from engineered modular type I polyketide synthases. Meth. Enzymol. 608, 393-415. doi:10.1016/bs.mie.2018.04.027
Zargar, A., Bailey, C. B., Haushalter, R. W., Eiben, C. B., Katz, L., Keasling, J. D., 2017. Leveraging microbial biosynthetic pathways for the generation of “drop-in” biofuels. Curr. Opin. Biotechnol. 45, 156-163. doi:10.1016/j.copbio.2017.03.004
Zargar, A., Barajas, J. F., Lal, R., Keasling, J. D., 2018. Polyketide synthases as a platform for chemical product design. AIChE J. doi:10.100²/aic.16351
Zargar, A.,et al., 2019. Chemoinformatic-guided engineering of polyketide synthases. BioRxiv. doi:10.1101/805671

TABLE S1

| Strains | Source |
|---|---|
| E. coli ET12567 | ATCC BAA-525 |
| S. venezuelae | ATCC 10712 |

Plasmids

| | | |
|---|---|---|
| Lip1 JuncA IdmO | JBx_078437 | Chimeric Lip1 with reductive loops swap from IdmO |
| | | at junction A with DEBS thioesterase |
| Lip1 JuncB IdmO | JBx_078438 | Chimeric Lip1 with reductive loops swap from IdmO |
| | | at junction B with DEBS thioesterase |
| Lip1 JuncA SpnB | JBx_078439 | Chimeric Lip1 with reductive loops swap from SpnB |
| | | at junction A with DEBS thioesterase |
| Lip1 JuncB SpnB | JBx_081781 | Chimeric Lip1 with reductive loops swap from SpnB |
| | | at junction B with DEBS thioesterase |
| Lip1 JuncB AurB | JBx_082096 | Chimeric Lip1 with reductive loops swap from AurB |
| | | at junction B with DEBS thioesterase |
| Lip1 JuncA AurB | JBx_082101 | Chimeric Lip1 with reductive loops swap from AurB |
| | | at junction A with DEBS thioesterase |
| Lip1 JuncB NanA2 | JBx_082097 | Chimeric Lip1 with reductive loops swap from NanA2 |
| | | at junction B with DEBS thioesterase |
| Lip1 JuncA NanA2 | JBx_081782 | Chimeric Lip1 with reductive loops swap from NanA2 |
| | | at junction A with DEBS thioesterase |
| Lip1 JuncB MAS | JBx_082098 | Chimeric Lip1 with reductive loops swap from MAS |
| | | at junction B with DEBS thioesterase |
| Lip1 JuncA MAS | JBx_081709 | Chimeric Lip1 with reductive loops swap from MAS |
| | | at junction A with DEBS thioesterase |
| Lip1 JuncA MonA2 | JBx_083535 | Chimeric Lip1 with reductive loops swap from MonA2 |
| | | at junction A with DEBS thioesterase |
| Lip1 JuncA LaidS2 | JBx_084029 | Chimeric Lip1 with reductive loops swap from LaidS2 |
| | | at junction A with DEBS thioesterase |
| Lip1 Native | JBx_082455 | Native Lip1 module |
| Lip2 JuncA SpnB | JBx_083532 | Chimeric Lip2 with reductive loops swap from SpnB |
| | | at junction A with DEBS thioesterase |
| Lip2 JuncA NanA2 | JBx_083533 | Chimeric Lip2 with reductive loops swap from NanA2 |
| | | at junction A with DEBS thioesterase |

### Supplemental Methods

### Cloning of Engineered LipPKS1 Reductive Loop Modules

The phiC31 Streptomyces integrase vectors were used as described by Phelan et al to integrate the LipPKS1 reductive loop swap modules²⁷. The native lipomycin module 1 with a fused DEBS thioesterase was used from Yuzawa et al³⁰. Reductive loop sequences of IdmO, AurB, NanA2, and SpnB sequences were codon optimized for E. coli and amplified from Hagen et al⁷. Reductive loop sequences from MAS, MonA2, and LaidS2 were codon optimized for E. coli and synthesized by Gen9 (since acquired by Ginkgo Bioworks). Cloning was performed through Golden Gate assembly. All clusters were expressed under the GapDH(El) promoter from Eggerthella lenta. Junction sites for reductive loop sites were determined by those reported by Hagen et al through multiple sequence alignment with Muscle³¹. The plasmids along with their associated information have been deposited in the public version of JBEI registry.

### Cloning of Native LipPKS1 and LipPKS2 Reductive Loop Modules

The phiC31 integrase vectors were used to integrate the native LipPKS1 module. The cloning of the native docking domain to replace the DEBS thioesterase was performed through Golden Gate assembly. The VWB Streptomyces integrase vectors were used as described by Phelan et al¹ to integrate the LipPKS2 reductive loop swap modules. The native LipPKS2 was codon optimized for E. coli and the native sequences synthesized with an attached DEBS thioesterase. Junction A reductive loop sites were determined through Muscle as in LipPKS1, and SpnB and NanA2 reductive loops were cloned through Golden Gate assembly. The plasmids along with their associated information have been deposited in the public version of JBEI registry.

Conjugation of phiC31 Integrase LipPKS1 Constructs into Streptomyces albus

E. coli ET12567/pUZ8002 was transformed with LipPKS1 plasmids and selected for on LB agar containing kanamycin (25 μg/mL), chloramphenicol (15 μg/mL), and apramycin (50 μg/mL). A single colony was used to inoculate a 5 mL of LB containing kanamycin (25 μg/mL), chloramphenicol (15 μg/mL), and apramycin (50 μg/mL) at 37° C. The overnight culture was used to seed 10 mL of LB containing the same antibiotics, which was grown at 37° C. to an OD600 of 0.4-0.6. The E. coli cells were pelleted by centrifugation, washed twice with LB, and resuspended in 500 μL of LB. Fresh S. albus J1074 spores were collected from a mannitol soy agar plate with 5 mL of 2×YT and incubated at 50° C. for 10 min. The spores (500 μL) and the E. coli cells (500 μL) were mixed, spread onto mannitol soy agar, and incubated at 30° C. for 16 hours. After 1 mL addition of nalidixic acid (20 μg/mL) and apramycin (40 μg/mL) was added and allowed to dry, the plate was further incubated for 3-4 days at 30° C. A single colony was used to inoculate into TSB containing nalidixic acid (25 μg/mL) and apramycin (25 μg/mL). After 3-4 days, a 1 mL aliquot was taken for genomic isolation through the Maxwell kit (Promega, Cat#AS1490, Madison Wis.). Successful integration was verified through qPCR. The remainder of the culture was spread onto a MS plate and incubated at 30° C. for 2-3 days. The spores were collected from the plate with 3-4 mL of water and mixed with glycerol to prepare 25% glycerol stock. The glycerol stock was stored at −80° C. for long-term storage.

Conjugation of VWB Integrase LipPKS2 Constructs into Streptomyces albus

E. coli ET12567/pUZ8002 was transformed with LipPKS2 plasmids and selected for on LB agar containing kanamycin (25 μg/mL), chloramphenicol (15 μg/mL), and spectinomycin (100 μg/mL). A single colony was used to inoculate a 5 mL of LB containing kanamycin (25 μg/mL), chloramphenicol (15 μg/mL), and apramycin (100 μg/mL) at 37° C. The overnight culture was used to seed 10 mL of LB containing the same antibiotics, which was grown at 37° C. to an OD600 of 0.4-0.6. The E. coli cells were pelleted by centrifugation, washed twice with LB, and resuspended in 500 μL of LB. S. albus J1074 spores with an integrated LipPKS1 were collected from a mannitol soy agar plate with 5 mL of 2×YT and incubated at 50° C. for 10 min. The spores (500 μL) and the E. coli cells (500 μL) were mixed, spread onto mannitol soy agar, and incubated at 30° C. for 16 hours. After a 1-mL addition of nalidixic acid (20 μg/mL), apramycin (40 μg/mL), and spectinomycin (400 μg/mL) was added and allowed to dry, the plate was further incubated for 3-4 days at 30° C. A single colony was used to inoculate into TSB containing nalidixic acid (25 μg/mL), apramycin (25 μg/mL) and spectinomycin (100 μg/mL). After 3-4 days, a 1-mL aliquot was taken for genomic isolation through the Maxwell kit (Promega, Cat#AS1490, Madison Wis.). Successful integration was verified through qPCR. The remainder of the culture was spread onto a MS plate and incubated at 30° C. for 2-3 days. The spores were collected from the plate with 3-4 mL of water and mixed with glycerol to prepare 25% glycerol stock. The glycerol stock was stored at -80° C. for long-term storage.

S. albus Production Runs

Engineered S. albus spores were grown in 12 mL of TSB medium containing nalidixic acid (50 μg/mL) and apramycin (50 μg/mL) for 4-5 days at 30° C. for single module LipPKS1 studies. Bimodular studies also included spectinomycin (200 μg/mL). 3 mL of the overnight culture was used to seed 30 mL of 10% media 042 and 90% plant hydrolysate⁵, supplemented with 2.4 grams/liter of valine and nalidixic acid (50 μg/mL), which was grown for 10 days at 30° C. For bimodular production runs, an overlay of 4 mL of dodecane was added to retain the triketide lactone.

### Sample Preparation for Single Module Production of Short Chain Acids

To detect β-keto, β-hydroxy, α-β alkene, and saturated acids, 1 mL of each sample was centrifuged at 5000×g for 10 minutes and 200 μL of the supernatant was removed. The supernatant was mixed with 200 μl of 100 μM hexanoic acid dissolved in methanol and filtered using Amicon Ultra Centrifugal filters, 3 kDa Ultracel, 0.5 mL device (Millipore). β-hydroxy (3-hydroxy-2,4-dimethylpentanoic acid) and saturated acids (2,4-dimethylpentanoic acid) were synthesized by Enamine (Cincinnati, USA) to greater than 95% purity.

### Sample Preparation for Bimodular Production of Triketide Lactones

Ten mL of each sample was mixed with 2 mL of diethyl ether in a 15 mL conical tube and vortexed for 5 minutes. Each conical tube was centrifuged at 5000 g for 10 minutes and 1 mL of ether was removed and placed into a 2 mL flat bottom microcentrifuge tube. Air was gently blown over each sample in a chemical fume hood until dry. The extract was resuspended in 200 μL of methanol with an internal standard of 100 μM δ-nonalactone (Sigma). 5-methyl-6-(propan-2-yl)oxan-2-one was synthesized by Enamine (Cincinnati, USA) to greater than 95% purity used as a standard to approximate 3,5-dimethyl-6-(propan-2-yl)oxan-2-one.

### Sample Preparation of Proteomics

Two milliliters of each production culture were harvested at 10 days, spun down at 10000 g for 10 minutes and resuspended in 0.75 mL of aqueous solution of 50 mM potassium phosphate, 300 mM NaCl, 10% glycerol and 2 mg/mL of lysozyme at pH 7.5. Samples were incubated for 1 hour at 30 degrees Celsius. Samples were then lysed with the ZR Fungal/Bacterial DNA Microprep kit (Zymo Research, Catalog No: D6007, Irvine, Calif.). Samples were loaded into the ZR Bashing/Bead Lysis tube and secured in a bead beater. Samples were shaken at a frequency of 30 per second for 5 minutes. The cell lysates were centrifuged at maximum speed in a Eppendorf benchtop centrifuge and the resulting supernatants were collected for proteomic analysis. The protein concentration of protein samples was determined by Bio-Rad DC Protein Assay (Bio-Rad #5000121) according to manufacture instruction. 20 ug protein of each sample was reduced and alkylated, followed by overnight trypsin digestion. The resulting tryptic peptide samples were subjected to LCMS analysis.

### LC-MS Detection of Short Chain Acids

LC separation of short-chain acids was conducted on an InfinityLab Poroshell HPH-C18 reversed phase column (100 mm length, 3.0 mm internal diameter, 2.7 pm particle size; Agilent, United States) using a Waters Acquity Autopurification system, prep UHPLC-MS (ESI) (Waters, United States). The mobile phase was composed of 10 mM ammonium acetate and 0.05% ammonium hydroxide in water (solvent A) and 10 mM ammonium acetate and 0.05% ammonium hydroxide in methanol (solvent B) to separate 2,4-dimethylpentanoic acid and 2,4-dimethylpent-2-enoic acid. The mobile phase was composed of 0.1% formic acid in water (solvent A) and 0.1%formic acid in methanol (solvent B) to separate 3-hydroxy-2,4-dimethylpentanoic acid and 2,3-dimethyl-3-oxopentanoic acid. All acids were each separated via the following gradient: increased from 5 to 97.1% B in 2.0 min, held at 97.1% B for 2.8 min, decreased from 97.1 to 5% B in 0.4 min, and held at 5% B for an additional 5.8 min. The flow rate was held at 0.42 ml·min−1 for 5.2 min, and then increased from 0.42 to 0.65 ml·min−1 for an additional 5.8 min. The total LC run time was 11 min. Samples were injected into the LC column at a volume of 15 μl. Acids were detected via [M−H]−ions: m/z=129.092; m/z=127.076; m/z=145.087; m/z=143.071. The analysis was performed using an m/z range of 70 to 500.

### LC-MS Detection of Triketide Lactones

LC separation of triketide lactones was conducted on a Kinetex XB-C18 reversed phase column (100 mm length, 3 mm internal diameter, 2.6 μm particle size; Phenomenex, United States) using an Agilent 1200 Rapid Resolution LC system (Agilent Technologies, United States). The mobile phase was composed of water (solvent A) and methanol (solvent B). Lactones were each separated via the following gradient: increased from 30 to 90% B in 3.7 min, held at 94% B for 5.2 min, decreased from 90 to 30% B in 0.33 min, and held at 30% B for an additional 2.0 min. The flow rate was held at 0.42 ml·min⁻¹ for 8.67 min, increased from 0.42 to 0.60 ml·min⁻¹ in 0.33 min, and held at 0.60 ml·min⁻¹ for an additional 2.0 min. The total LC run time was 11.0 min. The column compartment and autosampler temperatures were set to 50° C. and 6° C., respectively. Samples were injected into the LC column at a volume of 3 μl. The Agilent 1200 Rapid Resolution LC system was coupled to an Agilent 6210 TOF (Agilent Technologies, United States). Nitrogen gas was used as both the nebulizing and drying gas to facilitate the production of gas-phase ions. The drying and nebulizing gases were set to 101·min⁻¹ and 251·bin⁻², respectively, and a drying gas temperature of 325° C. was used throughout. Atmospheric pressure chemical ionization was conducted in the positive-ion mode with capillary and fragmentor voltages of 3.5 kV and 100 V, respectively. The skimmer, OCT1 RF, and corona needle were set to 50 V, 170 V, and 4 μA, respectively. The vaporizer was set to 350° C. Lactones were detected via [M+H]⁺ ions: m/z=157.1223; m/z=171.1379. The analysis was performed using an m/z range of 70 to 1100. Data acquisition and processing were performed using MassHunter software (Agilent Technologies, US).

### Chemical Synthesis

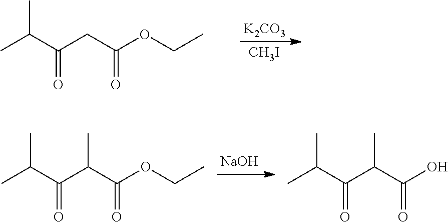

Synthesis of 2,4-dimethyl-3-oxopentanoic acid. A solution of ethyl 4-methyl-3-oxopentanoate (1.5 g, 9.5 mmol), potassium carbonate (3.93 g, 28.5 mmol) and methyl iodide (1.62 g, 11.4 mmol) in THF (20 mL) was refluxed overnight under a N2 environment. The reaction mixture was allowed to cool to room temperature before subsequent filtration and concentration was carried out, resulting in the synthesis of ethyl 2,4-dimethyl-3-oxopentanoate as clear yellow oil. Without further purification, the oil was dissolved in 10 mL 1N NaOH aqueous solution and stirred overnight at room temperature. The solution was acidified to pH 1-2 using conc. HCl and the resulting mixture was extracted with diethyl ether (30 mL×3). The combined organic layers were dried over sodium sulfate, filtered and concentrated in vacuo to give the crude product, which was purified by flash column chromatography (silica, DCM: EA 1:1) to yield 2,4-dimethyl-3-oxopentanoic acid a pale-yellow oil (985 mg, 72% over two steps). ¹H NMR (400 MHz, Chloroform-d) δ 4.06 (q, J=7.1 Hz, 1H), 2.74 (hept, J=6.9 Hz, 1H), 1.21 (d, J=7.1 Hz, 3H), 1.01 (dt, J=6.9, 2.5 Hz, 6H).

### Proteomic Analysis

Shotgun proteomic was analyzed on an Agilent 6550 iFunnel Q-TOF mass spectrometer (Agilent Technologies, Santa Clara, Calif.) coupled to an Agilent 1290 UHPLC system as described previously³². Peptides were separated on a Sigma—Aldrich Ascentis Peptides ES-C18 column (2.1 mm×100 mm, 2.7 μm particle size, operated at 60° C.) at a 0.400 mL/min flow rate and eluted with the following gradient: initial condition was 95% solvent A (0.1% formic acid) and 5% solvent B (99.9% acetonitrile, 0.1% formic acid). Solvent B was increased to 35% over 120 min, and then increased to 50% over 5 min, then up to 90% over 1 min, and held for 7 min at a flow rate of 0.6 mL/min, followed by a ramp back down to 5% B over 1 min where it was held for 6 min to re-equilibrate the column to original conditions. Peptides were introduced to the mass spectrometer from the LC by using a Jet Stream source (Agilent Technologies) operating in positive-ion mode (3,500 V). Source parameters employed gas temp (250° C.), drying gas (14 L/min), nebulizer (35 psig), sheath gas temp (250° C.), sheath gas flow (11 L/min), VCap (3,500 V), fragmentor (180 V), OCT 1 RF Vpp (750 V). The data were acquired with Agilent MassHunter Workstation Software, LC/MS Data Acquisition B.06.01 operating in Auto MS/MS mode whereby the 20 most intense ions (charge states, 2-5) within 300-1,400 m/z mass range above a threshold of 1,500 counts were selected for MS/MS analysis. MS/MS spectra (100-1,700 m/z) were collected with the quadrupole set to “Medium” resolution and were acquired until 45,000 total counts were collected or for a maximum accumulation time of 333 ms. Former parent ions were excluded for 0.1 min following MS/MS acquisition. The successfully identified peptides of engineered LipPKS1 were targeted in a SRM method developed on an Agilent 6460 QQQ mass spectrometer system coupled with an Agilent 1290 UHPLC system (Agilent Technologies, Santa Clara, Calif.). Peptides were separated on an Ascentis Express Peptide C18 column [2.7-mm particle size, 160-Å pore size, 5-cm length×2.1-mm inside diameter (ID), coupled to a 5-mm×2.1-mm ID guard column with the same particle and pore size, operating at 60° C.; Sigma-Aldrich] operating at a flow rate of 0.4 ml/min via the following gradient: initial conditions were 98% solvent A (0.1% formic acid), 2% solvent B (99.9% acetonitrile, 0.1% formic acid). Solvent B was increased to 40% over 11 min, and was then increased to 80% over 1 min, and held for 1.5 min at a flow rate of 0.6 mL/min, followed by a ramp back down to 2% B over 0.5 min where it was held for 1 min to re-equilibrate the column to original conditions. The eluted peptides were ionized via an Agilent Jet Stream ESI source operating in positive ion mode with the following source parameters: gas temperature=250° C., gas flow=13 liters/min, nebulizer pressure=35 psi, sheath gas temperature=250° C., sheath gas flow=11 liters/min, capillary voltage=3500 V, nozzle voltage=0 V. The data were acquired using Agilent MassHunter version B.08.02. Acquired SRM data were analyzed by Skyline software version 20.1 (MacCoss Lab Software).

### Phylogenetic Trees

Amino acid sequences of KR and KS domains from 72 BGCs were extracted from ClusterCAD¹⁷. The sequences were aligned using Muscle v3.8³³. The alignments were manually curated using JalView³⁴. The best amino acid substitution model for both the ketosynthases and ketoreductases phylogenies was LG+F+I+G4, and it was selected using the ModelFinder tool implemented in IQ-tree³⁵. Finally the phylogeny was constructed using IQ-tree³⁶, using the partitioned models with 10,000 bootstrap replicates and visualized with FigTree.

### Statistics

Spearman rank correlations were used to compare the significance of reductive loop exchanges. Production titers were normalized to the highest producer in the production of short chain acids in LipPKS1 swaps and triketide lactone production in LipPKS2 swaps. The datasets were combined and production titer and chemosimilarity were normalized to the highest production titer and most chemically similar reductive loop. The datasets were then tested for statistical significance through a Spearman correlation.

### SUPPLEMENTAL REFERENCES

1. Phelan, R. M. et al. Development of Next Generation Synthetic Biology Tools for Use in Streptomyces venezuelae. ACS Synth. Biol. 6, 159-166 (2017).
2. Yuzawa, S. et al. Heterologous Gene Expression of N-Terminally Truncated Variants of LipPks1 Suggests a Functionally Critical Structural Motif in the N-terminus of Modular Polyketide Synthase. ACS Chem. Biol. 12, 2725-2729 (2017).
3. Hagen, A. et al. Engineering a polyketide synthase for in vitro production of adipic acid. ACS Synth. Biol. 5, 21-27 (2016).
4. Madeira, F. et al. The EMBL-EBI search and sequence analysis tools APIs in 2019. Nucleic Acids Res. 47, W636—W641 (2019).
5. González Fernández-Niño, S. M. et al. Standard flow liquid chromatography for shotgun proteomics in bioenergy research. Front. Bioeng. Biotechnol. 3, 44 (2015).
6. Eng, C. H. et al. ClusterCAD: a computational platform for type I modular polyketide synthase design. Nucleic Acids Res. 46, D509-D515 (2018).
7. Edgar, R. C. MUSCLE: multiple sequence alignment with high accuracy and high throughput. Nucleic Acids Res. 32, 1792-1797 (2004).
8. Waterhouse, A. M., Procter, J. B., Martin, D. M. A., Clamp, M. & Barton, G. J. Jalview Version 2—a multiple sequence alignment editor and analysis workbench. Bioinforrnatics 25, 1189-1191 (2009).
9. Kalyaanamoorthy, S., Minh, B. Q., Wong, T. K. F., von Haeseler, A. & Jermiin, L. S. ModelFinder: fast model selection for accurate phylogenetic estimates. Nat. Methods 14, 587-589 (2017).
10. Nguyen, L.-T., et al. IQ-TREE: a fast and effective stochastic algorithm for estimating maximum-likelihood phylogenies. Mol. Biol. Evol. 32, 268-274 (2015).

What is claimed is:

1. A composition comprising a microbe comprising an engineered polyketide synthase (PKS) configured to programmably produce a lactone, wherein the PKS is engineered with a first acyltransferase (AT) swap and a reductive loop (RL) swap.

2. The composition of claim 1, wherein the lactone is a delta lactone, the PKS is lipomycin (Lip) PKS, the LipPKS comprises a first LipPSK1 module comprising the first AT swap, and a second LipPSK2 module comprising the RL swap.

3. The composition of claim 2, wherein the second LipPSK2 module further comprises a second AT swap.

4. The composition of claim 1, wherein the lactone is a dimethylated delta-lactone.

5. The composition of claim 1, wherein the lactone is a single-methylated delta-lactone.

6. The composition of claim 1, wherein the lactone is a nonmethylated delta-lactone.

7. The composition of claim 1, wherein the first AT swap is a borrelidin (Bor) AT swap.

8. The composition of claim 1, wherein the RL swap is a nanchangamycin (NanA2) RL swap.

9. The composition of claim 3, wherein the second AT swap is a borrelidin (Bor) AT swap.

10. The composition of claim 1, wherein the first AT swap is a borrelidin (Bor) AT swap, and the RL swap is a nanchangamycin (NanA2) RL swap, to programmably produce a single-methylated delta lactone.

11. The composition of claim 1, wherein the first AT swap is a borrelidin (Bor) AT swap, the RL swap is a nanchangamycin (NanA2) RL swap, and the second LipPSK2 module further comprises a second AT swap, to programmably produce a non-methylated delta lactone.

12. The composition of claim 2, wherein a malonyl-CoA selecting analog is employed in the first and second module, a KR only in the first module, and a full reductive loop in the second module.

13. The composition of claim 1, wherein the microbe is Streptomyces albus.

14. A method of making a delta-lactone comprising incubating the composition of claim 1 under conditions wherein the microbes produce the delta lactone.

15. A method of making a delta-lactone comprising engineering lipomycin PKS to produce the delta-lactone.

16. The method of claim 15: wherein the lactone is a dimethylated delta-lactone, single-methylated delta-lactone, or nonmethylated delta-lactone; comprising in a first module, performing an acyltransferase (AT) swap with a BorAT and in a second module performing a reductive loop swap with a NanA2 to programmably produce a single-methylated delta lactone; comprising in a first module, performing an acyltransferase (AT) swap with a BorAT and in a second module performing a reductive loop swap with a NanA2, and another AT swap in the second module to programmably produce a non-methylated delta lactone; and/or wherein a malonyl-CoA selecting analog is employed in the first and second module, a KR only in the first module, and a full reductive loop in the second module.

17. A composition comprising an engineered lipomycin PKS1 gene (or gene product) altered with an AT-swap from borreledin and a LipPKS2 altered with a donor reductive loop from NanA2, configured to produce a single-methylated lactone.

18. The composition of claim 17 comprising another AT swap on LipPKS2 from borreledin, configured to produce a non-methylated delta lactone.
